(19) United States
(12) Patent Application Publication
(10) Pub. No.: US 2026/0191750 A1
(43) Pub. Date: Jul. 9, 2026

(54) WAX ESTERS

(71) Applicant: Evonik Operations GmbH, Essen (DE)

(72) Inventors: Jan Marian von Hof, Bochum (DE); Renate Maier, Witten (DE); Christian Hartung, Essen (DE); Sven Klare, Essen (DE); Matthias Mentel, Dortmund (DE); Achim Friedrich, Bochum (DE); Steffen Tekstra, Borken (DE)

(73) Assignee: Evonik Operations GmbH, Essen (DE)

(21) Appl. No.: 19/132,952

(22) PCT Filed: May 22, 2023
(86) PCT No.: PCT/EP2023/082637

§ 371 Date: May 27, 2025

(30) Foreign Application Priority Data
Nov. 30, 2022 (EP) 22210499.4

(51) Int. Cl. A61K 8/36; A61K 8/34; A61Q 17/04; C11C 1/02

(52) U.S. Cl. CPC A61K 8/36; A61K 8/345; A61Q 17/04; C11C 1/025

(57) ABSTRACT

A process produces optionally at least partially saponified wax esters and corresponding products are obtainable by the process. Wax esters produced by the process find application in topical applications.

### FIELD OF THE INVENTION

The invention relates to a process for producing optionally at least partially saponified wax esters, to the corresponding products obtainable by the process of the invention, and to the use thereof in topical applications.

### PRIOR ART

Natural waxes are a class of waxes divided into the subgroups of plant waxes, animal waxes and mineral waxes.

Examples include cotton wax, carnauba wax, candelilla wax, esparto wax, guaruma wax, japan wax, cork wax, montan wax, ouricury wax, rice germ oil wax, sugarcane wax and also beeswax, preen gland fat, wool wax, shellac wax and spermaceti, as well as microwaxes, ceresin and ozokerite.

The oxidation of such natural waxes affords wax oxidates, also referred to synonymously as acid waxes.

DE102018116113A1 describes the production of a natural wax oxidate from rice bran wax and/or sunflower wax having an acid value of >45 mg KOH/g to <70 mg KOH/g and a process for the production thereof.

DE102013007638 A1 describes a process for producing acid waxes/wax oxidates having an acid value of 100-160 mg KOH/g obtained by chromosulfuric acid oxidation of mixtures of natural wax esters (rice husk wax, carnauba wax, sunflower wax or sugarcane wax) and α-olefins and (partially saponified) esters of such acid waxes with monoethylene glycol and butanediol and the use thereof for the production of shoe polish.

DE10231886A1 describes carnauba wax oxidates and a process for the production thereof. Repeated treatment with chromosulfuric acid allows almost white carnauba wax oxidates having increased acid values of ≥60, ≥80 preferably ≥100 mg, KOH/g to be obtained in the described process, through at least partial cleavage of the wax esters and oxidation of the alcohols present to the corresponding carboxylic acids after purification with dilute sulfuric acid and water. The composition of a carnauba acid wax (=carnauba wax oxidate) thereby obtained is stated as: wax monocarboxylic acids (approx. 59%), wax dicarboxylic acids (approx. 4%) and wax esters (approx. 33%). The wax monocarboxylic acids in the carnauba wax oxidate have chains having even and odd numbers of carbon atoms ranging from C13 to C34. Monocarboxylic acids having 32 (16.5%) and 24 (11.1%) carbon atoms are dominant, with monocarboxylic acids containing 20, 22, 26, 28 and 30 carbon atoms (in each case approx. 6-7%) also present in similar amounts. In addition, the C16 and C18 monocarboxylic acids are present in equal amounts (4.3%) and the other monocarboxylic acids in amounts of approx. 2-3% each.

WO2014060081A1 discloses rice bran wax oxidates having an acid value of at least 70 mg KOH/g, preferably at least 100 mg KOH/g, more preferably at least 140 mg KOH/g, as raw materials for chemical derivatizations for the production of synthetic ester waxes and the use thereof for the production of (semi)synthetic and partially saponified ester waxes by esterification with one or more monovalent or polyvalent alcohols. Rice bran waxes consist mainly of monoesters of long-chain saturated unbranched fatty acids with long-chain unbranched aliphatic alcohols. C22 and C24 predominate in the acid fraction, whereas C26, C28, C30, C32 and C34 are predominant in the alcohol fraction. Here, chromic acid oxidation results in high acid values when the rice bran wax had previously been hydrolysed under harsh alkaline conditions. The described rice bran wax oxidate comprises free C16 to C36 carboxylic acids, C24 being the principal component, with significant amounts of C22 and C24 also present. The described rice bran wax oxidate also contains small amounts (5-15%) of aliphatic α,ω-dicarboxylic acids (C10 to C32). Examples of uses include additives in plastics processing (internal and external lubricants, parting agents, mould-release agents, dispersants for pigments), as constituents of care products (pastes, polishes, emulsions) or cosmetic preparations, as additives for printing inks, for abrasion protection, as an additive for lacquers for matting or for improving scratch resistance.

DE2450342 describes mixed oxidates of bark wax, the esterification of such mixed oxidates with ethylene glycol and the use of such products for producing self-shine emulsions for floors, for producing cleaning agents for shoes, as a lubricant and parting agent for the processing of polyvinyl chloride and also for producing emulsions for hydrophobizing chipboard.

DE102013003366A1 describes dimethylaminopropylamides of oxidates of natural waxes and the use thereof in cosmetic compositions, especially hair-care products.

DE102014001709A1 describes combinations of cationic hair treatments with dimethylaminopropylamides of oxidates of natural waxes and the use thereof in cosmetic preparations, especially hair-care products.

Commercially available cosmetic film formers for sun protection and make-up formulations are mostly high-molecular-weight synthetic/petrochemically based, non-biodegradable polymers such as polyurethanes, polyacrylates, polyolefins, polyvinylpyrrolidones or the corresponding copolymers thereof. Examples thereof are products with the Antaron/Ganex (Ashland), Baycusan (Covestro) or TEGO® SP (Evonik) trade names.

Reasonably hydrophilic and thus reasonably readily water-soluble/water-swellable film formers based on natural polymers have likewise been described and are based for example on polysaccharides such as starch, cellulose and derivatives thereof or polypeptides. However, these do not exhibit good water resistance and often also show inadequate sun protection factor (SPF) boosting properties in sun protection or make-up formulations.

Products based on renewable raw materials are of ever-increasing interest to consumers not just from an environmental perspective, but from a toxicological perspective, too. For instance, partial esters of polyols with fatty acids have already made inroads into diverse fields of use in cosmetics and other areas. Corresponding products having “film-forming properties” are already on the market. Examples of these include:
Syncrowax™ ORM (Croda; INCI: Sorbitol/Sebacic Acid Copolymer Behenate),
Cera Bellina #106 (Koster Keunen; INCI: Polyglyceryl-3 Beeswax),
CosmoSurf® PG1-IS (Surfatech Corp.; INCI: Polyglyceryl-3 Stearate/Isostearate Dimer Dilinoleate Copolymer),
SurfaCare S (Surfatech Corp.; INCI: Oleic/Linoleic/Linolenic Polyglycerides),
LexFilm Sun natural (Inolex; INCI: Capryloyl Glycerin/Sebacic Acid Copolymer),
SolAmaze™ Natural polymer (Nouryon; INCI: Diisostearoyl Polyglyceryl-3 Dimer Dilinoleate (and) Caprylic/Capric Triglyceride),
Pelemol 6GPR (Phoenix Chem; INCI: Polyglyceryl-6 Polyricinoleate) and
Nomcort HK-G (INCI: Glyceryl Behenate/Eicosadioate), Nomcort HK-P (INCI: Polyglyceryl-10 Behenate/Eicosadioate) and Nomcort SG (INCI: Glyceryl Tribehenate/Isostearate/Eicosadioate) from Nisshin Oillio Group.

However, all these products have inadequate film-forming properties compared to the abovementioned synthetic/petrochemical-based polymers.

Esters of polyglycerols are a particularly interesting group of products on account of the diverse possibilities for variation:

WO2018033259A1 discloses polyglycerol esters obtainable by esterification of a polyglycerol with a carboxylic acid mixture comprising at least one polyhydroxycarboxylic acid of a hydroxycarboxylic acid having 8 to 32 carbon atoms, at least one short-chain dicarboxylic acid having 2 to 16 carbon atoms, at least one long-chain dicarboxylic acid having 24 to 44 carbon atoms and at least one fatty acid selected from linear, unsaturated and branched, saturated fatty acids having 14 to 24 carbon atoms.

The object of the invention was to provide excellent film formers, especially for use in sun protection formulations.

### DESCRIPTION OF THE INVENTION

Surprisingly, it has been found that the wax esters described hereinbelow are able to achieve the object of the invention.

The present invention accordingly provides a process for producing an optionally at least partially saponified wax ester.

The present invention further provides the product obtainable by the process of the invention and formulations comprising it.

The present invention further provides for the use of the wax esters of the invention.

An advantage of the present invention is that the wax esters described herein may be prepared exclusively from renewable raw materials, unlike the polyacrylates described above.

A further advantage of the present invention is that the wax esters described herein can be prepared on the basis of principles of green chemistry.

Another advantage of the present invention is that formulations can be provided that are polyglycol ether-free.

A further advantage of the present invention is that the wax esters described herein are biodegradable, unlike the polyacrylates described above.

A further advantage is that the wax esters described herein have a good ecotoxicological profile.

A further advantage of the wax esters according to the invention is that they are very mild on the skin, not irritating and non-toxic.

Another advantage of the wax esters according to the invention is that they have improved sensory properties in formulations. The tackiness of sun protection formulations is reduced after application.

The formulations are absorbed more rapidly into the skin, since absorption is increased during application and for 5 min thereafter.

Another advantage of the wax esters according to the invention is that they give rise to enhanced “velvety-silkiness” in formulations.

A further advantage of the present invention is that the wax esters described herein have a good skin-moisturizing effect.

Another advantage of the present invention is that the wax esters described herein impart an increased sun protection factor to sun protection formulations.

Another advantage is that the wax esters described herein are very well suited to use in sun protection formulations having very high concentrations of UV light protection filters.

A further advantage of the wax esters according to the invention is that they impart very high water resistance to formulations. In sun protection formulations this results in the formulations ensuring prolonged UV protection in water or after bathing.

Another advantage of the wax esters according to the invention is that they impart increased wear resistance to colour pigments when used in make-up applications.

In the context of pigment-containing formulations, a further advantage of the wax esters according to the invention is that they permit good dispersion of pigments in the formulations.

A further advantage of the wax esters according to the invention is that they have good compatibility with formulations containing UV protection filters or pigments.

In general, the wax esters described herein impart good stability to the formulations.

Another advantage of the wax esters according to the invention is that they are easily processable, since they mix readily with typical cosmetic oils and can be rapidly incorporated into corresponding emulsions.

A further advantage of the wax esters according to the invention is that they impart high gloss to solid or waxy formulations such as lipsticks.

Another advantage of the wax esters according to the invention is that they are particularly tolerant to electrolytes, which means that, for example, formulations containing large amounts of salt remain stable.

A further advantage of the wax esters according to the invention is that they have a structuring and viscosity-increasing effect in formulations having high oil contents or even pure oils.

The present invention provides a process for producing an optionally at least partially saponified wax ester, comprising the process steps of:
A) providing a natural wax,
B) oxidizing the natural wax to a natural wax oxidate,
C) esterifying the natural wax oxidate with at least one polyol, optionally additionally with at least one selected from monocarboxylic acids and polycarboxylic acids, especially dicarboxylic acids, and optionally
D) at least partially saponifying the wax ester obtained in process step C),
   with the proviso that, if only one polyol is used in process step C), said one polyol has a hydroxyl value of less than 1240 mg KOH/g, preferably less than 1200 mg KOH/g, more preferably less than 1150 mg KOH/g.

Suitable methods for determining the hydroxyl value are in particular those according to DGF C-V 17 a (53), Ph. Eur. 2.5.3 Method A and DIN 53240.

Unless stated otherwise, all stated percentages (%) are percentages by mass.

The provided natural wax is preferably obtained from renewable raw material sources and especially ones of nonfossil/petrochemical origin. Examples of such natural waxes from renewable sources are alfalfa wax, bamboo wax, cotton wax, beeswax, candelilla wax, caranday wax, carnauba wax, dammar wax, Douglas fir wax, esparto wax, flax wax, hemp wax, coffee wax, cork wax, oleander wax, ouricury wax, raffia wax, rice wax, rice bran wax, retamo wax, bark wax, sisal wax, tea wax, wool wax and sugarcane wax, with carnauba wax particularly preferred.

The provided natural wax preferably has a high content of wax esters. The proportion of corresponding fatty oil constituents (for example rice bran oil in the case of rice wax) should be not more than 50% by weight, preferably not more than 30% by weight, more preferably not more than 5% by weight, based on the total natural wax.

Preferably, the provided natural wax contains in total 0.05% by weight to 35% by weight, preferably 0.1% by weight to 25% by weight, more preferably 1.0% by weight to 20% by weight, of aliphatic ω-hydroxycarboxylic acids and/or aliphatic α,ω-alkanediols and/or esters of aliphatic ω-hydroxycarboxylic acids and/or esters of aliphatic α,ω-alkanediols, where the percentages by weight refer to the total natural wax.

Preferably, the aliphatic monocarboxylic acids present in the wax esters of the provided natural wax have a chain length of C20 or greater to an extent of 10% by weight to 98% by weight, preferably 20% by weight to 95% by weight, more preferably 30% by weight to 90% by weight, based on all aliphatic monocarboxylic acids present.

Preferably, the ω-hydroxycarboxylic acids present in the wax esters of the provided natural wax have a chain length of C24 or greater to an extent of 0.1% by weight to 50% by weight, preferably 1% by weight to 45% by weight, more preferably 10% by weight to 40% by weight, based on all ω-hydroxycarboxylic acids present.

The provided natural wax may also contain phospholipids, sterol derivatives, sterol esters, oryzanols, tocotrieneols, glycolipids, and/or squalenes, more particularly in an amount totalling less than 12% by weight, preferably less than 5% by weight, more preferably less than 2% by weight, especially preferably less than 1% by weight, based on the total weight of the provided natural wax.

In process step B) of the process according to the invention, the provided natural wax is preferably oxidized with at least partial cleavage of the wax esters present in the provided natural wax. During the oxidation, the primary alcohols present in the reaction mixture are at least partially oxidized to the corresponding carboxylic acids.

Preferably, process step B) of the process according to the invention is carried out over two or more stages, for example initially via at least partial saponification and/or—preferably alkaline—hydrolysis and subsequent oxidation.

The process for the oxidation of the natural wax in process step B) of the process according to the invention can take place in one or more stages. The oxidation process may be carried out in the presence of oxidation promoters (preferably not more than 10% by weight, preferably not more than 5% by weight, more preferably not more than 1% by weight, based on the total reaction mixture). Oxidation promoters, by virtue of their chemical nature, accelerate oxidation through phase transfer, ester activation or by catalysis. Suitable examples include those described in WO2014060082A1 and the literature cited therein.

Preferably, the oxidation process in process step B) of the process according to the invention is carried out with mechanical dispersion (for example with ultrasound, dissolver disk, etc.).

Preferably, the oxidation in process step B) of the process according to the invention is carried out with chromosulfuric acid.

Alternative methods to chromosulfuric acid oxidation that are likewise highly suitable are described for example in WO2020025813A1 (oxidation by oxoammonium cations and co-oxidants) and WO2020025814A1 (preliminary saponification and subsequent oxidation by aminoxyl radicals and co-oxidants such as chlorites and bromites).

When a metal-catalysed oxidation has been carried out in process step B) of the process according to the invention, it is preferable that metal soaps present are, prior to further reaction, removed by washing (for example with aqueous solutions of oxalic acid and sulfuric acid) or centrifuging.

In the specific case of chromium salts, the purity of the wax oxidates of the invention preferably meets the German Pharmacopoeia specifications for Cera montanglycoli.

In process step B) of the process according to the invention, two or more natural waxes may be provided.

It is also possible in process step B) of the process according to the invention to add to the provided natural wax additional other esters, diesters, paraffins, mono- and polyfunctional carboxylic acids, fatty alcohols, aldehydes and/or other substances that afford corresponding oxidates under the prevailing conditions.

As a result thereof, the chain distributions of aliphatic monocarboxylic acids and aliphatic α,ω-dicarboxylic acids in a mixed oxide thus obtained can differ significantly from those of a pure natural wax oxidate.

The cleavage of the wax esters and subsequent oxidation of the hydroxyl groups of the wax alcohols or ω-hydroxycarboxylic acids or the oxidative cleavage of unsaturated constituents (for example unsaturated carboxylic acids) results in an increase in the saponification value (SV) of the natural wax oxidates thus obtained.

In accordance with the invention, it is preferable that the natural wax oxidate obtained in process step B) of the process according to the invention has a saponification value of from 60 to 260 mg KOH/g, preferably 80 to 240 mg KOH/g, more preferably from 100 to 220 mg KOH/g.

Suitable methods for determining the saponification value are in particular those according to DGF C-V 3, DIN EN ISO 3681 and Ph. Eur. 2.5.6.

The proportion of genuine wax esters, that is to say esters untouched by the cleavage of the wax esters and subsequent oxidation, is preferably less than 65% by weight, even more preferably less than 50% by weight, especially preferably less than 40% by weight, particularly preferably less than 30% by weight, where the percentages by weight refer to the total natural wax oxidate.

In accordance with the invention, it is preferable that the natural wax oxidate obtained in process step B) of the process according to the invention has an acid value (AV) of from 30 to 200 mg KOH/g, preferably 50 to 190 mg KOH/g, more preferably from 70 to 180 mg KOH/g.

Suitable methods for determining the acid value are in particular those according to DGF C-V 2, DIN EN ISO 2114, Ph. Eur. 2.5.1, ISO 3682 and ASTM D 974.

In accordance with the invention, it is preferable that the natural wax oxidate obtained in process step B) of the process according to the invention has the characteristic feature of a very low content of unsaturated and/or aromatic compounds. Accordingly, the natural wax oxidate obtained in process step B) of the process according to the invention preferably has an iodine value (IV) of ≤40 g I₂/100 g, preferably of ≤20 g I₂/100 g, more preferably of ≤10 g I₂/100 g, especially preferably of ≤5 g I₂/100 g.

A suitable method for determining the iodine value in the context of the present invention is EN 14111:2003.

In accordance with the invention, it is preferable that the natural wax oxidate obtained in process step B) of the process according to the invention is characterized by a drip point measured according to DIN ISO 2176 of between 40° C. and 130° C., preferably between 50° C. and 105° C., even more preferably between 70° C. and 90° C., especially preferably between 75° C. and 87° C.

In accordance with the invention, it is preferable that the natural wax oxidate obtained in process step B) of the process according to the invention has a content of aliphatic monocarboxylic acids having a chain length of 8 to 34 carbon atoms of 30-90% by weight, preferably of 40-80% by weight, even more preferably of 50-70% by weight, where the percentages by weight refer to all aliphatic monocarboxylic acids and aliphatic α,ω-dicarboxylic acids present in the natural wax oxidate.

In accordance with the invention, it is preferable that the natural wax oxidate obtained in process step B) of the process according to the invention has a content of aliphatic α,ω-dicarboxylic acids having a chain length of 8 to 34 carbon atoms of 1% by weight to 40% by weight, preferably of 2% by weight to 35% by weight, even more preferably of 20% by weight to 30% by weight, where the percentages by weight refer to all aliphatic monocarboxylic acids and α,ω-dicarboxylic acids present in the natural wax oxidate. This proportion of aliphatic α,ω-dicarboxylic acids can originate either directly from the natural wax (for example by cleavage and in-situ oxidation of esters of aliphatic α,ω-diols present, cleavage and in-situ oxidation of esters of ω-hydroxycarboxylic acids present or oxidative cleavage of unsaturated constituents, for example oleic esters or similar) and/or by cleavage and in-situ oxidation of added components (oils, glycerides, etc., see below) and/or they can be added prior to further reaction (for example addition of sebacic acid or similar).

In accordance with the invention, it is preferable that the natural wax oxidate obtained in process step B) of the process according to the invention has a total content of aliphatic α,ω-dicarboxylic acids having a chain length of 8 to 34 carbon atoms and aliphatic monocarboxylic acids having a chain length of 8 to 34 carbon atoms of more than 60% by weight, preferably of more than 70% by weight, even more preferably of more than 80% by weight, where the percentages by weight refer to the total natural wax oxidate.

In accordance with the invention, it is preferable that the natural wax oxidate obtained in process step B) of the process according to the invention has a content of aliphatic monocarboxylic acids having a chain length of 20 or more carbon atoms of at least 40% by weight, preferably of at least 60% by weight, even more preferably of at least 80% by weight, where the percentages by weight refer to all aliphatic monocarboxylic acids present in the natural wax oxidate.

In accordance with the invention, it is preferable that the natural wax oxidate obtained in process step B) of the process according to the invention has a content of aliphatic monocarboxylic acids having an odd-numbered chain length of 9 to 29 carbon atoms of at least 0.01% by weight, preferably of at least 0.5% by weight, even more preferably of at least 5.0% by weight, especially preferably of at least 10.0% by weight, where the percentages by weight refer to all aliphatic monocarboxylic acids present in the natural wax oxidate.

In accordance with the invention, it is preferable that the natural wax oxidate obtained in process step B) of the process according to the invention has a content of aliphatic α,ω-dicarboxylic acids having a chain length of 12 to 29 carbon atoms of more than 50% by weight, preferably of more than 60% by weight, even more preferably of more than 80% by weight, where the percentages by weight refer to all aliphatic α,ω-dicarboxylic acids present in the natural wax oxidate.

In accordance with the invention, it is preferable that the natural wax oxidate obtained in process step B) of the process according to the invention has a content of aliphatic α,ω-dicarboxylic acids having a chain length of 18 or more carbon atoms of more than 20% by weight, preferably of more than 40% by weight, even more preferably of more than 60% by weight, where the percentages by weight refer to all aliphatic α,ω-dicarboxylic acids present in the natural wax oxidate.

In accordance with the invention, it is preferable that the natural wax oxidate obtained in process step B) of the process according to the invention has a content of aliphatic α,ω-dicarboxylic acids having an odd-numbered chain length of 9 to 29 carbon atoms of at least 0.01% by weight, preferably of at least 0.5% by weight, even more preferably of at least 5.0% by weight, especially preferably of at least 10.0% by weight, where the percentages by weight refer to all aliphatic α,ω-dicarboxylic acids present in the natural wax oxidate.

The abovementioned percentages by weight of the aliphatic monocarboxylic acids and α,ω-dicarboxylic acids are always determined as a relative GC area percentage according to the method described below:

The distributions of aliphatic monocarboxylic acids and α,ω-dicarboxylic acids in samples to be analysed can be determined via GC.

This method involves derivatization of the wax oxidates and subsequent simultaneous determination by GC/FID. If the distributions of aliphatic monocarboxylic acids and α,ω-dicarboxylic acids in wax esters are to be determined, the wax esters need to first undergo alkaline hydrolysis. For this, an initial charge of 150 mg of the wax ester to be analysed in 2.00 ml of 1 M aqueous KOH solution is hydrolysed at 95° C. with stirring for 30 min. The reaction solution is then cooled to room temperature and adjusted to pH 2-3 with 2 M aqueous HCl solution. The carboxylic acids that precipitate out as a result are then extracted with diethyl ether (3×3.00 ml) and the extract concentrated to dry by evaporation.

0.05 g of such an extract or of the wax oxidate is then dissolved to a clear solution in 10 ml of toluene with heating to 60° C. 0.25 ml of the still-warm, clear solution is mixed with 0.5 ml of BSTFA [N,O-bis(trimethylsilyl)trifluoroacetamide] and 0.05 ml of TMCS [trimethylchlorosilane]. The aliphatic monocarboxylic acids and aliphatic α,ω-dicarboxylic acids are at 80° C. quantitatively converted into their trimethylsilyl esters within 30 minutes and then analysed by GC/FID.

This is performed in a gas chromatograph equipped with an on-column injector, a capillary column and a flame-ionization detector, under the following conditions:
Injector: CoC, Track Oven
Injected volume: 0.5 μl
Column: 30 m*0.32 mm DB5-HT 0.1 μm
Carrier gas: Hydrogen, const. flow, 2 ml/min
Temperature program: 65° C. at 10° C./min to 365° C.
   then conditioning at 365° C. for 15 minutes.
Detector: FID at 365° C.
   Hydrogen 35 ml/min
   Air 240 ml/min
   Make-up gas 12 ml/min

The aliphatic monocarboxylic acids and aliphatic α,ω-dicarboxylic acids present in the samples are separated according to chain length. The peaks are assigned by comparison with the retention times of the corresponding commercially available aliphatic monocarboxylic acids and aliphatic α,ω-dicarboxylic acids.

The relative proportions of the individual acids can be determined by evaluating the respective peak areas.

In process step C), the natural wax oxidate is esterified with at least one polyol, with the proviso that if only one polyol is used in process step C), said one polyol has a hydroxyl value of less than 1240 mg KOH/g, preferably less than 1200 mg KOH/g, more preferably less than 1150 mg KOH/g. Suitable reaction conditions for process step C) are temperatures between 120° C. to 260° C. and atmospheric pressure or else reduced pressure within a range between 20 mbar to 800 mbar, especially between 50 and 500 mbar. Process step C) can be carried out in the presence of catalytic amounts of a base, selected in particular from metal hydroxides, metal oxides and metal carbonates such as NaOH, Ca(OH)₂, KOH, Zn(OH)₂, CaO, ZnO, Na₂CO₃, CaCO₃ or K₂CO₃ or catalytic amounts of an acid, especially sulfuric acid, phosphoric acid, phosphinic acid, methanesulfonic acid, ethanesulfonic acid or para-toluenesulfonic acid. Corresponding methods can be found in standard chemistry textbooks such as Römpp.

A preferred process according to the invention is characterized in that the at least one polyol has three or more, preferably four or more, especially more than six, carbon atoms. Where a plurality of polyols is used, it goes without saying that the mixture should have an average of three or more, preferably four or more, especially more than six, carbon atoms.

Where a plurality of polyols is used, it is in accordance with the invention preferable that this mixture has a hydroxyl value of less than 1240 mg KOH/g, preferably less than 1200 mg KOH/g, more preferably less than 1150 mg KOH/g.

A preferred process according to the invention is characterized in that the at least one polyol comprises polyglycerol, wherein the polyglycerol accounts for preferably at least 20% by weight, preferably at least 40% by weight, even more preferably at least 60% by weight, especially preferably at least 80% by weight, based on all polyols.

The term “polyglycerol” is for the purposes of the present invention to be understood as meaning a polyglycerol that may also contain glycerol. Consequently, when calculating amounts, masses and the like, any glycerol fraction should also be taken into consideration. Its polymeric character means that polyglycerol is a statistical mixture of various compounds. Polyglycerol may have ether bonds between two primary, one primary and one secondary, or else two secondary positions of the glycerol monomers. The polyglycerol base framework accordingly does not usually consist exclusively of linearly linked glycerol units but may also comprise branchings and rings. For details, see for example “Original synthesis of linear, branched and cyclic oligoglycerol standards”, Cassel et al., J. Org. Chem. 2001, 875-896.

It is preferable in accordance with the invention that the average degree of polymerization of the polyglycerol used in step C) is 2 to 20, preferably 2.5 to 16, and most preferably 3 to 12.

For the calculation, the average degree of polymerization of the polyglycerol <n> is calculated via the hydroxyl value (OHV, in mg KOH/g) according to the formula <n>=(112200-18*OHV)/(74*OHV-56100).

A preferred process according to the invention is characterized in that the polyglycerol present in the at least one polyol has a content of cyclic oligomers, i.e. oligomers containing one or more rings, of from 1.0% by weight to 50% by weight, preferably from 2.0% by weight to 40% by weight, more preferably from 3.0% by weight to 30% by weight.

Further polyols preferably used in process step C) of the process according to the invention are selected from the group:
decane-11,10-diol, dodecane-1,12-diol, hexane-1,2-diol, octane-1,2-diol, 1,2-pentylene glycol, 1,4-bis(hydroxymethyl)cyclohexane, pentane-1,5-diol, 2,2,4-trimethylpentane-1,3-diol, 2,2-bis(4-hydroxycyclohexyl)propane, 2,2-dimethylpropane-1,3-diol (neopentyl glycol), 2,4-diethylpentane-1,5-diol, 2,5-dimethyl-3-hexyne-2,5-diol, 2-butyl-2-ethylpropane-1,3-diol, 2-ethylhexane-1,3-diol, 2-methylpropane-1,3-diol, 2-methyl-2-propylpropane-1,3-diol, 2-sec-butyl-2-methylpropane-1,3-diol, 3-hexyne-2,5-diol, ditrimethylolpropane, isosorbide, pinacol, tricyclodecanedimethanol and tripentaerythritol.

Further polyols used with preference in process step C) of the process according to the invention together with at least one further polyol are selected from the group:
1,2-Butylene glycol, 1,2-propylene glycol, 1,3-butylene glycol, propane-1,3-diol, butane-1,4-diol, 1,4-sorbitan, 1,5-sorbitan, diglycerol, dipentaerythritol, erythritol, glycerol, isomalt, lactitol, maltitol, mannitol, pentaerythritol, sorbitol, trimethylolethane, trimethylolpropane, xylitol and xylitol.

In process step C) of the process according to the invention, the natural wax oxidate is optionally additionally esterified with at least one selected from monocarboxylic acids and polycarboxylic acids, especially dicarboxylic acids, more preferably selected from aliphatic α,ω-dicarboxylic acids having a chain length of 8 to 34 carbon atoms and aliphatic monocarboxylic acids having a chain length of 8 to 34 carbon atoms.

This can be achieved by simple addition by acyl group donors of said acids, for example from other natural waxes, fats, oils, fatty acid partial esters or also the acids themselves.

In the optional process step D) of the process according to the invention, the wax ester obtained in process step C) is at least partially saponified.

For this purpose, water and preferably basic metal hydroxides, such as NaOH, KOH, Ca(OH)₂ and Zn(OH)₂, metal oxides, such as CaO, metal carbonates, such as Na₂CO₃ and CaCO₃, and/or aqueous alkalis are added to the wax ester and the mixture thus obtained is saponified and optionally then dried under reduced pressure, especially at temperatures above the melting point of the wax ester concerned, preferably between 70-100° C., preferably with stirring.

The present invention further provides an optionally at least partially saponified wax ester obtainable by the process according to the invention.

These have the surprising property of being able to structure oils particularly well and also of boosting the UV filtering effect of light protection filters.

Preferred optionally at least partially saponified wax esters of the present invention correspond in their degree of preference to the corresponding preferred processes of the invention by which they are obtainable.

These optionally at least partially saponified wax esters of the invention have an excellent use profile in cosmetic formulations.

The present invention accordingly further provides a formulation, for topical applications in particular, especially a cosmetic formulation comprising the optionally at least partially saponified wax esters of the invention.

The formulations according to the invention comprise with corresponding preference the optionally at least partially saponified wax esters of the invention shown above as preferred.

Preferably, the formulation according to the invention additionally comprises at least one substance selected from the group comprising UV light protection filter substances and pigments, especially UV light protection filter substances.

The UV light protection filter substances present may for example be organic substances that are capable of absorbing ultraviolet radiation and re-emitting the absorbed energy in the form of longer-wavelength radiation, for example heat.

UVB filters can be oil-soluble or water-soluble. Examples of oil-soluble UVB light protection filters include:
3-benzylidenecamphor derivatives, for example 3-(4-methylbenzylidene)camphor (INCI: 4-Methylbenzylidene Camphor), 4-aminobenzoic acid derivatives, for example 2-ethylhexyl 4-(dimethylamino)benzoate (INCI: Ethylhexyl Dimethyl PABA), 2-hydroxyethyl 4-{bis[2-(2-hydroxyethoxy)ethyl]amino}benzoate (INCI: PEG-25 PABA), cinnamic esters, for example 2-ethylhexyl 4-methoxycinnamate (INCI: Ethylhexyl Methoxycinnamate), isopentyl 4-methoxycinnamate (INCI: Isoamyl p-Methoxycinnamate), 2-ethylhexyl 2-cyano-3,3-diphenylacrylate (INCI: Octocrylene), salicylic esters, for example 2-ethylhexyl salicylate (INCI: Ethylhexyl Salicylate), homomenthyl salicylate (INCI: Menthyl Salicylate), 3,3,5-trimethylcyclohexyl salicylate (INCI: Homosalate), 2-hydroxybenzoic acid-2,2′,2″-nitrilotriethanol (1:1) (INCI: TEA-Salicylate), benzophenone derivatives, for example 2-hydroxy-4-methoxybenzophenone (INCI: Benzophenone-3), 2,2′-dihydroxy-4-methoxybenzophenone (INCI: Benzophenone-8), 2-hydroxy-4-methoxy-4′-methylbenzophenone (INCI: Benzophenone-10), triazine derivatives, for example tris(2-ethylhexyl) 4,4′,4″-(1,3,5-triazine-2,4,6-triyltriimino)tribenzoate (INCI: Ethylhexyl Triazone, obtainable for example under the trade name Uvinul T 150 from BASF), iscotrizinol (INCI: Diethylhexyl Butamido Triazone) and 2,4,6-tri(4-biphenylyl)-1,3,5-triazine (INCI: Tris-Biphenyl Triazine).

An additional UVB filter is the (3-(4-(2,2-bis(ethoxycarbonyl)vinyl)phenoxy)propenyl)methoxysiloxane/dimethylsiloxane copolymer (INCI: Polysilicone-15) obtainable for example under the trade name Parsol SLX.

Useful water-soluble UVB sun protection filters include for example:
salts of 2-phenylbenzimidazole-5-sulfonic acid, such as its alkali metal, alkaline earth metal, ammonium, alkylammonium, alkanolammonium and glucammonium salts, and also the sulfonic acid itself having the INCI name Phenylbenzimidazole Sulfonic Acid, sulfonic acid derivatives of benzophenone, for example 5-benzoyl-4-hydroxy-2-methoxybenzenesulfonic acid (INCI: Benzophenone-4) and salts thereof, benzenesulfonic acid derivatives of 3-benzylidenecamphor, for example 4-[(E)-(4,7,7-trimethyl-3-oxobicyclo[2.2.1]hept-2-ylidene)methyl]benzenesulfonic acid (INCI: Benzylidene Camphor Sulfonic Acid) and salts thereof.

Examples of typical oil-soluble UVA/broadband light protection filters that may be used include derivatives of benzoylmethane, for example 1-(4-methoxyphenyl)-3-[4-(2-methyl-2-propanyl)phenyl]-1,3-propanedione (INCI: Butyl Methoxydibenzoylmethane) or 1-(4-isopropylphenyl)-3-phenyl-1,3-propanedione (INCI: Isopropyl Dibenzoylmethane), triazine derivatives, for example 2,2′-[6-(4-methoxyphenyl)-1,3,5-triazine-2,4-diyl]bis{5-[(2-ethylhexyl)oxy]phenol}(INCI: Bis-Ethylhexyloxyphenol Methoxyphenyl Triazine, obtainable for example under the trade name Tinosorb S from BASF), N,N′-bis[4-[5-(1,1-dimethylpropyl)-2-benzoxazolyl]phenyl]-N″-(2-ethylhexyl)-1,3,5-triazine-2,4,6-triamine (INCI: Ethylhexyl Bis-Isopentylbenzoxazolylphenyl Melamine, obtainable under the trade name Uvasorb K2A from 3V Sigma), and derivatives of benzophenone, for example hexyl 2-(4-diethylamino-2-hydroxybenzoyl)benzoate (INCI: Diethylamino Hydroxybenzoyl Hexyl Benzoate).

Useful water-soluble UVA/broadband light protection filters include, for example: 3,3′-(1,4-phenylenedimethylene)-bis(7,7-dimethyl-2-oxobicyclo[2.2.1]hept-1-ylmethanesulfonic acid) and salts thereof, especially the corresponding sodium, potassium or triethanolammonium salt, which is also described as benzene-1,4-di(2-oxo-3-bornylidenemethyl-10-sulfonic acid) and has the INCI name Terephthalylidene Dicamphor Sulfonic Acid (obtainable under the trade name Mexoryl SX), 2,2′-(1,4-phenylene)-bis(6-sulfo-1H-benzimidazole-4-sulfonic acid) and salts thereof, the corresponding sodium, potassium or triethanolammonium salts, for example disodium 2,2′-(1.4-phenylene)-bis(6-sulfo-1H-benzimidazole-4-sulfonate) having the INCI name Disodium Phenyl Dibenzimidazole Tetrasulfonate, trade name for example Neo Heliopan AP.

Examples of other UVA/broadband filters are 2,2′-methylenebis[6-(2H-benzotriazol-2-yl)-4-(2,4,4-trimethyl-2-pentanyl)phenol](INCI: Methylene Bis-Benzotriazolyl Tetramethylbutylphenol, obtainable for example under the trade name Tinosorb M from BASF), 2-(2H-benzotriazol-2-yl)-4-methyl-6-[2-methyl-3-[1,3,3,3-tetramethyl-1-[(trimethylsilyl)oxy]disiloxanyl]propyl]phenol (INCI: Drometrizole Trisiloxane, trade name: Mexoryl XL), (1R,2S,5R)-2-isopropyl-5-methylcyclohexyl 2-aminobenzoate (INCI: Menthyl Anthranilate) and 2-ethoxyethyl (2E)-3-(4-methoxyphenyl)acrylate (INCI: Cinoxate).

The UV light protection filter substances may of course also be present in mixtures in the compositions according to the invention.

In addition to the soluble UV light protection filter substances mentioned, insoluble pigments may also be used for this purpose, namely finely dispersed metal oxides or salts, for example titanium dioxide, zinc oxide, iron oxide, aluminium oxide, cerium oxide, zirconium oxide, silicates (talc), barium sulfate and zinc stearate. The particles should here have an average diameter of less than 100 nm, for example of between 5 and 50 nm and especially between 15 and 30 nm. They may be spherical in shape, but it is also possible to use particles that are ellipsoidal in shape or have a shape that deviates in another way from spherical. Also possible however are micronized organic pigments, for example 2,2′-methylenebis[6-(2H-benzotriazol-2-yl)-4-(2,4,4-trimethyl-2-pentanyl)phenol](INCI: Methylene Bis-Benzotriazolyl Tetramethylbutylphenol, obtainable for example under the trade name Tinosorb M from BASF), having a particle size of <200 nm, which is obtainable for example as a 50% aqueous dispersion.

In addition, further suitable UV light protection filter substances can be found in the review by P. Finkel in SÖFW Journal, 122, 543 (1996) and in the chapter “The Chemistry of Ultraviolet Filters” by N. A. Shaath in “Principles and Practice of Photoprotection”, S. Q. Wang and H. W. Lim (eds.), Springer International Publishing, Switzerland, 2016.

It is preferable in accordance with the invention that the formulation according to the invention comprises at least two, preferably at least three, more preferably at least four, UV light protection filter substances.

It is preferable in accordance with the invention that the at least one UV light protection filter substance in the formulation according to the invention is selected from the group comprising organic UV light protection filter substances, especially from the group of triazine derivatives.

It is particularly preferable in accordance with the invention that the at least one UV light protection filter substance in the formulation according to the invention is selected from the group of UV light protection filter substances comprising, preferably consisting of,
Butyl Methoxydibenzoylmethane,
Ethylhexyl Triazone,
Bis-Ethylhexyloxyphenol Methoxyphenyl Triazine,
Diethylhexyl Butamido Triazone,
Diethylamino Hydroxybenzoyl Hexyl Benzoate,
Benzophenone-3,
Benzophenone-4,
4-Methylbenzylidene Camphor,
Octocrylene,
Ethylhexyl Methoxycinnamate,
Ethylhexyl Salicylate,
Homomenthyl Salicylate,
Phenylbenzimidazole Sulfonic Acid,
Methylene Bis-Benzotriazolyl Tetramethylbutylphenol,
Disodium Phenyl Dibenzimidazole Tetrasulfonate,
Isoamyl p-Methoxycinnamate,
Ethylhexyl Dimethyl PABA.

It is in addition preferable in accordance with the invention that the at least one UV light protection filter substance in the formulation according to the invention is selected from the group of pigments comprising unmodified or surface-modified, inorganic or organic pigments, preferably inorganic pigments such as activated carbon, talc, iron oxide pigments, titanium dioxide, zinc oxide, silica, cerium oxides, zirconium oxides, aluminium oxides, calcium carbonate, barium sulfate, calcium sulfate, alkali magnesium silicates or mixtures thereof.

Examples of organic pigments may be: carotenoids, chlorophylls, xanthophylls, caramel and the salts of the recited examples and also colorants obtained from fruits of plants or other plant parts, for example from orange, cacao, turmeric, shea, sandalwood, onion, carob tree fruit, paprika, maize, tomato, beetroot, peanut, grape, red cabbage, red rice, radish, elderberry, lingonberry, blueberry, raspberry, blackberry, boysenberry, gooseberry, cranberry, saffron, strawberry, cherry, tea, hibiscus, plum, blueberry or mulberry. Also employable as colouring pigments are for example the substances approved and suitable for cosmetic purposes, as listed for example in the publication “Kosmetische Färbemittel” [Cosmetic colorants] of the Dyes Committee of the Deutsche Forschungsgemeinschaft [German Research Foundation], Verlag Chemie, Weinheim, 1984, pages 81 to 106.

It is in addition preferable in accordance with the invention when the formulation according to the invention comprises at least one selected from the group of UV light protection filter substances, said formulation additionally comprising pigments selected from titanium dioxide and zinc oxide. In this connection too, the UV light protection filter substances that are preferably present correspond to the preferred UV light protection filter substances mentioned above.

A preferred formulation according to the invention is characterized in that the optionally at least partially saponified wax ester according to the invention is present in the formulation according to the invention in an amount of from 0.1% by weight to 20% by weight, preferably from 0.25% by weight to 12% by weight, more preferably from 0.5% by weight to 6% by weight, where the percentages by weight refer to the total formulation.

A preferred formulation according to the invention is characterized in that the at least one UV light protection filter substance in the formulation according to the invention is present in an amount of from 0.1% by weight to 60% by weight, preferably from 1% by weight to 50% by weight, more preferably from 10% by weight to 40% by weight, where the percentages by weight refer to the total formulation.

The formulations according to the invention may comprise for example at least one further, additional component selected from the group comprising emollients,
co-emulsifiers,
thickeners/viscosity regulators/stabilizers,
antioxidants,
hydrotropes (or polyols),
solids and fillers,
pearlescent additives and opacifiers
insect repellents,
self-tanning agents,
preservatives,
conditioning agents,
perfumes,
colorants,
cosmetic active substances,
care additives,
superfatting agents,
solvents.

Substances that can be used as exemplary representatives of the individual groups are known to those skilled in the art and can for example be taken from German application DE 102008001788.4. This patent application is hereby incorporated by reference and is thus considered to form part of the disclosure.

As regards further optional components and also the amounts used of these components, reference is expressly made to the relevant handbooks known to those skilled in the art, for example K. Schrader, “Grundlagen und Rezepturen der Kosmetika” [Fundamentals and formulations of cosmetics], 2nd edition, pages 329 to 341, Hüthig Buch Verlag, Heidelberg.

The amounts of the respective additives depend on the intended use.

Typical starting formulations for the relevant applications are known prior art and are contained for example in the brochures of the manufacturers of the relevant base materials and active substances. These existing formulations can generally be adopted unchanged. However, any desired modifications necessary for adjustment and optimization can be made in a straightforward manner through simple tests.

Formulations according to the invention may for example be used in the form of an emulsion, a suspension, a solution, a cream, a salve, a paste, a gel, an oil, a powder, an aerosol, a stick, a spray, a cleansing product, a make-up product or a sun protection product.

The present invention further provides for the use of the optionally at least partially saponified wax esters of the present invention for producing a formulation, especially for topical applications, especially a cosmetic formulation, especially a sun protection formulation.

The present invention further provides for the use of the optionally at least partially saponified wax esters of the present invention as film formers.

The present invention further provides for the use of the optionally at least partially saponified wax esters of the present invention for boosting the sun protection factor of a UV light protection filter substance.

The present invention further provides for the use of the optionally at least partially saponified wax esters of the present invention for reducing the rinseability and/or wear of a formulation from a surface.

In the use according to the invention, preference is given to using, as components that are preferably present, the components mentioned above in the context of the formulations according to the invention.

The examples that follow describe the present invention by way of example without any intention to limit the invention, the scope of application of which is apparent from the entirety of the description and the claims, to the embodiments specified in the examples.

### EXAMPLES

### Example 001 (Inventive)

A mixture of commercially available polyglycerol-3 from Spiga Nord (OHV=1100 mg KOH/g, 50.0 g) and commercially available natural wax oxidate CEVO®-cos K-4419 from Voelpker (AV=138 mg KOH/g, SV=172 mg KOH/g, 295.9 g) was heated to 240° C. with stirring and through-passage of N₂ and the water that formed was continuously distilled off until an acid value of 2.5 mg KOH/g was attained. The melting point of the product was 83° C., the saponification value was 159 mg KOH/g.

### Example 002 (Inventive)

A mixture of commercially available polyglycerol-3 from Spiga Nord (OHV=1100 mg KOH/g, 30.3 g) and commercially available natural wax oxidate CEVO®-cos K-4419 from Voelpker (AV=138 mg KOH/g, SV=172 mg KOH/g, 89.7 g) was heated to 240° C. with stirring and through-passage of N₂ and the water that formed was continuously distilled off until an acid value of 2.1 mg KOH/g was attained. The melting point of the product was 80° C., the saponification value was 139 mg KOH/g.

### Example 003 (Inventive)

A mixture of commercially available polyglycerol-3 from Spiga Nord (OHV=1100 mg KOH/g, 50.0 g) and commercially available natural wax oxidate CEVO®-cos K-4419 from Voelpker (AV=138 mg KOH/g, SV=172 mg KOH/g, 74.0 g) was heated to 240° C. with stirring and through-passage of N₂ and the water that formed was continuously distilled off until an acid value of 1.2 mg KOH/g was attained. The melting point of the product was 80° C., the saponification value was 109 mg KOH/g.

### Example 004 (Inventive)

A mixture of commercially available polyglycerol-3 from Spiga Nord (OHV=1100 mg KOH/g, 21.4 g) and commercially available natural wax oxidate CEVO®-cos K-4419 from Voelpker (AV=138 mg KOH/g, SV=172 mg KOH/g, 98.6 g) was heated to 240° C. with stirring and through-passage of N₂ and the water that formed was continuously distilled off until an acid value of 2.5 mg KOH/g was attained. The melting point of the product was 80° C., the saponification value was 151 mg KOH/g.

### Example 005 (Inventive)

A mixture of commercially available polyglycerol-6 from Spiga Nord (OHV=989 mg KOH/g, 80.6 g) and commercially available natural wax oxidate CEVO®-cos K-4419 from Voelpker (AV=138 mg KOH/g, SV=172 mg KOH/g, 119.4 g) was heated to 240° C. with stirring and through-passage of N₂ and the water that formed was continuously distilled off until an acid value of 2.3 mg KOH/g was attained. The melting point of the product was 80° C., the saponification value was 109 mg KOH/g.

### Example 006 (Inventive)

A mixture of commercially available polyglycerol-6 from Spiga Nord (OHV=989 mg KOH/g, 50.5 g) and commercially available natural wax oxidate CEVO®-cos K-4419 from Voelpker (AV=138 mg KOH/g, SV=172 mg KOH/g, 149.5 g) was heated to 240° C. with stirring and through-passage of N₂ and the water that formed was continuously distilled off until an acid value of 2.3 mg KOH/g was attained. The melting point of the product was 80° C., the saponification value was 138 mg KOH/g.

### Example 007 (Inventive)

A mixture of commercially available polyglycerol-3 from Spiga Nord (OHV=1116 mg KOH/g, 34.0 g), commercially available natural wax oxidate CEVO®-cos K-4419 from Voelpker (AV=138 mg KOH/g, SV=172 mg KOH/g, 100.8 g) and behenic acid (92%, AV=165 mg KOH/g, 65.2 g) was heated to 240° C. with stirring and through-passage of N₂ and the water that formed was continuously distilled off until an acid value of 2.0 mg KOH/g was attained. The melting point of the product was 72° C., the saponification value was 159 mg KOH/g.

### Example 008 (Inventive)

A mixture of commercially available polyglycerol-3 from Spiga Nord (OHV=1116 mg KOH/g, 49.1 g), commercially available natural wax oxidate CEVO®-cos K-4419 from Voelpker (AV=138 mg KOH/g, SV=172 mg KOH/g, 145.5 g) and sebacic acid (99%, 5.4 g) was heated to 240° C. with stirring and through-passage of N₂ and the water that formed was continuously distilled off until an acid value of 0.7 mg KOH/g was attained. The melting point of the product was 83° C., the saponification value was 150 mg KOH/g.

### Examples 16a-i (Noninventive)

a. TEGO® SP 13 Sun Up MB (Evonik, INCI: Poly C10-30 Alkyl Acrylate)
b. Antaron V-220 (Ashland, INCI: VP/Eicosene Copolymer)
c. ISOLAN® PDI (Evonik, INCI: Diisostearoyl Polyglyceryl-3 Dimer Dilinoleate)
d. ISOLAN® GPS (Evonik, INCI: Polyglyceryl-4 Diisostearate/Polyhydroxystearate/Sebacate)
e. Cera Bellina #106 (Koster Keunen; INCI: Polyglyceryl-3 Beeswax),
f. CosmoSurf PG1-IS (Surfatech Corp.; INCI: Polyglyceryl-3 Stearate/Isostearate Dimer Dilinoleate Copolymer),
g. LexFilm™ Sun Natural MB (Inolex, INCI: Capryloyl Glycerin/Sebacic Acid Copolymer)
h. Polyglycerol esters according to example 1 from WO2018033259
i. Polyglycerol esters according to example 14 from WO2018033259

### Application Example 101: In-Vitro SPF-Boosting Test of Cosmetic Formulations

Wax esters according to the invention have the characteristic feature of a strong tendency to film formation compared to noninventive esters or noninventive commercial products marketed as film formers. The film-forming properties in sun protection formulations can be quantitatively demonstrated by means of in-vitro SPF tests.

This was done by preparing O/W sun protection emulsions on a 200 g laboratory scale into which were incorporated the various film formers in an each time identical manner according to the recipes hereinbelow (Table1). The emulsions were prepared according to methods known to those skilled in the art commonly used in the production of lotions.

TABLE 1
Composition of the O/W sun protection emulsions
for in-vitro determination of the SPF

| | | Formulation | |
|---|---|---|---|
| Phase | Ingredient | A (reference) | Formulation B |
| A | TEGO ® Care PBS 6 MB (Polyglyceryl-6 Stearate, | 3.00% | 3.00% |
| | Polyglyceryl-6 Behenate) | | |
| | TEGIN ® M Pellets MB (Glyceryl Stearate) | 0.50% | 0.50% |
| | TEGO ® Alkanol 1618 (Cetearyl Alcohol) | 0.50% | 0.50% |
| | TEGOSOFT ® XC MB (Phenoxyethyl Caprylate) | 5.00% | 3.50% |
| | Bis-Ethylhexyloxyphenol Methoxyphenyl Triazine | 3.00% | 3.00% |
| | Butyl Methoxydibenzoylmethane | 2.00% | 2.00% |
| | Ethylhexyl Salicylate | 3.00% | 3.00% |
| | Octocrylene | 8.00% | 8.00% |
| | Homosalate | 3.00% | 3.00% |
| | Ethylhexyl Triazone | 1.50% | 1.50% |
| | dermofeel ® Toco 70 non GMO | 0.70% | 0.70% |
| | (Tocopherol, Helianthus Annuus (Sunflower) | | |
| | Seed Oil) | | |
| | Inventive wax esters from examples 001-008 / | — | 1.50% |
| | noninventive examples 16a-i | | |
| B | Glycerin | 3.00% | 3.00% |
| | Disodium EDTA | 0.05% | 0.05% |
| | Water | to 100% | to 100% |
| C | Xanthan Gum (Keltrol CG-SFT, CP Kelco) | 0.50% | 0.50% |
| D | Verstatil ® PC (Phenoxyethanol, Caprylyl Glycol) | 1.20% | 1.20% |

24.5 mg of reference formulation A or of test formulations B was respectively evenly applied (1 mg/cm²) and spread over polymethyl methacrylate plates (PMMA; 7.0 cm×3.5 cm, 2 μm roughness, Schonberg GmbH & Co. KG). For each measurement, six replicate determinations were carried out, i.e. each formulation was applied to 6 individual PMMA plates. These were left in a drying cabinet at 30° C. for 30 min, after which the SPF of the plates was determined using a Labsphere UV2000S Ultraviolet Transmittance Analyzer in accordance with Colipa Guideline (2011) with in each case 4 measurement points per PMMA plate. The individual SPF values for each PMMA plate/formulation were noted and the mean value determined. The mean SPF values for each formulation with film former were divided by the mean SPF value of formulation A without film former. The results are listed below in Table 2.

TABLE 2
Relative in-vitro SPF values of the
example products in formulation B

| | | SPF boost |
|---|---|---|
| | | (relative to |
| | | reference |
| Formulation | | formulation |
| B | Samples present in formulation B | A) |
| B1 | TEGO ® SP 13 Sun Up | 1.94 |
| | (Poly C10-30 Alkyl Acrylate), example 16a | |
| B2 | Antaron V-220 | 1.55 |
| | (Ashland, INCI: VP/Eicosene Copolymer), | |
| | example 16b | |
| B3 | ISOLAN ® PDI | 1.15 |
| | (Diisostearoyl Polyglyceryl-3 Dimer | |
| | Dilinoleate), example 16c | |
| B4 | ISOLAN ® GPS | 1.16 |
| | (Polyglyceryl-4 Diisostearate/ | |
| | Polyhydroxystearate/Sebacate), example 16d | |
| B5 | Polyglyceryl-3 Beeswax | 1.39 |
| | (Cera Bellina, Koster Keunen), example 16e | |
| B6 | Polyglyceryl-3 Stearate/Isostearate/Dimer | 1.01 |
| | Dilinoleate Crosspolymer | |
| | (CosmoSurf PG1-IS, SurfaTech Corp.), | |
| | example 16f | |
| B7 | Capryloyl Glycerin/Sebacic Acid Copolymer | 1.00 |
| | (LexFilm Sun Natural MB, Inolex Inc.), | |
| | example 16g | |
| B8 | Polyglycerol esters according to example | 1.11 |
| | 1 from WO2018033259, example 16h | |
| B9 | Polyglycerol esters according to example | 1.29 |
| | 14 from WO2018033259, example 16i | |
| B10* | Ex. 001 | 2.00 |
| B11* | Ex. 002 | 1.98 |
| B12* | Ex. 003 | 1.41 |
| B13* | Ex. 004 | 1.68 |
| B14* | Ex. 005 | 1.66 |
| B15* | Ex. 006 | 2.32 |
| B16* | Ex. 007 | 2.05 |
| B17* | Ex. 008 | 2.20 |

*inventive examples

The results in Table 2 show for the inventive examples in all cases SPF-boost values of over 1.40 (relative to reference formulation A without film former). In fact, the best wax esters according to the invention achieved values better than those with the polyacrylate TEGO® SP 13 Sun Up and with the synthetic VP/Eicosene Copolymer. Values significantly better than those of plant-based reference substance of the prior art were obtained.

### Application Example 102: Oil Structuring

A mixture of 5 g of the respective wax ester according to the invention or of two noninventive examples and 95 g of various (cosmetic) oils was stirred at 70° C. for 30 min, then stored at 25° C. for 24 h and the nature of the mixtures thus obtained examined. The results are listed below in Table 3.

TABLE 3
Oil-structuring capability

| | | Cera Bellina #106 | |
|---|---|---|---|
| | TEGO ® SP 13 Sun Up | (Koster Keunen, INCI: | |
| | MB (Evonik, INCI: Poly | Polyglyceryl-3 | |
| | C10-30 Alkyl Acrylate, | Beeswax), | All examples 001 to |
| | example 16a), | example 16e), | 008, |
| | noninventive | noninventive | all inventive |
| Sunflower oil | Solid, not free-flowing | Liquid, free-flowing | Solid, not free-flowing |
| Mineral oil | Solid, not free-flowing | Liquid, free-flowing | Solid, not free-flowing |
| 2-Ethylhexyl | Solid, not free-flowing | Liquid, free-flowing | Solid, not free-flowing |
| palmitate | | | |

The results show that inventive examples 001 to 008 are just as effective at thickening oils as the polyacrylate TEGO® SP 13 Sun Up based on petrochemical raw materials (example 16a), whereas the plant-based reference substance Cera Bellina #106 (example 16e) does not show any oil structuring.

### Formulation Examples

The formulation examples below demonstrate the usability of the inventive wax esters in cosmetic emulsions by way of example and do not limit the subject matter of the invention.

All percent values are unless otherwise stated percentages by weight. Production and homogenization steps are carried out according to the usual methods.

If necessary, the pH is adjusted with acids or bases, this being noted accordingly in the formulation. Since the amount of acid or base needed can depend on the batches of the other ingredients, it is in the examples here often entered as q.s. (=quantum satis). It is also necessary to adjust to different pH values depending on the preservative used. The customary pH values that were used and adjusted to here in the example formulations are in the range from pH 3.5 to 8.0.

The example recipes listed hereinbelow were in each case produced with each of the inventive wax esters from examples 001 to 008 (referred to below as “Example X”); for each example formulation, eight different formulations were thus prepared.

### Fun in the Sun SPF 30 Spray

| | | % | % |
|---|---|---|---|
| Phase | Raw material | w/w | w/w |
| A | TEGO ® Care PBS 6 MB (Polyglyceryl-6 | 3.00 | 3.00 |
| | Stearate, Polyglyceryl-6 Behenate) | | |
| | dermofeel ® sensolv MB (Isoamyl Laurate) | 3.00 | 3.00 |
| | Bis-Ethylhexyloxyphenol Methoxyphenyl | 3.00 | 3.00 |
| | Triazine (Tinosorb S, BASF) | | |
| | Butyl Methoxydibenzoylmethane | 2.00 | 2.00 |
| | Homosalate | 4.00 | 4.00 |
| | Ethylhexyl Salicylate | 4.00 | 4.00 |
| | Octocrylene | 4.00 | 4.00 |
| | Example X | 0.50 | 1.50 |
| | VP/Eicosene Copolymer (Antaron V-220, | 0.50 | |
| | Ashland) | | |
| | TEGO ® Feel C 10 (Cellulose) | 1.00 | 1.00 |
| B | Water | to 100 | to 100 |
| | Gellan Gum (Kelcogel CG-HA, CP Kelco) | 0.03 | 0.03 |
| | EDTA | 0.05 | 0.05 |
| | Glycerin | 3.00 | 3.00 |
| | Acrylates/C10-30 Alkyl Acrylate Crosspolymer | 0.20 | 0.20 |
| C | Phenylbenzimidazole Sulfonic Acid | 2.00 | 2.00 |
| | Tromethamine | 0.88 | 0.88 |
| | Water | 7.12 | 7.12 |
| D | Tromethamine (Trisaminomethane, 30% in | q.s. | q.s. |
| | water) | | |
| E | dermofeel ® OMP (Methylpropanediol, | 3.00 | 3.00 |
| | Caprylyl Glycol, Phenylpropanol) | | |

### Oil Release Sun Care Lotion SPF 50

| | | % | % |
|---|---|---|---|
| Phase | Raw material | w/w | w/w |
| A | TEGO ® Care PBS 6 MB (Polyglyceryl-6 | 3.00 | 3.00 |
| | Stearate, Polyglyceryl-6 Behenate) | | |
| | TEGOSOFT ® XC (Phenoxyethyl Caprylate) | 15.00 | 15.00 |
| | TEGOSOFT ® DC MB (Decyl Cocoate) | 10.00 | 10.00 |
| | TEGOSOFT ® CT (Caprylic/Capric | 10.00 | 10.00 |
| | Triglyceride) | | |
| | Example X | 1.50 | 2.50 |
| | Diethylamino Hydroxybenzoyl Hexyl | 10.00 | 10.00 |
| | Benzoate (Uvinul A plus, BASF) | | |
| | Ethylhexyl Methoxycinnamate | 10.00 | 10.00 |
| | Bis-Ethylhexyloxyphenol Methoxyphenyl | 5.00 | 5.00 |
| | Triazine (Tinosorb S, BASF) | | |
| | Ethylhexyl Salicylate | 5.00 | 5.00 |
| | Homosalate | 5.00 | 5.00 |
| | TEGO ® Feel C 10 (Cellulose) | 1.00 | — |
| | Dimethicone Crosspolymer | — | 0.50 |
| B | Water | to 100 | to 100 |
| | Glycerin | 3.00 | 3.00 |
| C | Verstatil ® TBO (Triethyl Citrate; | 1.20 | 1.20 |
| | Caprylyl Glycol; Benzoic Acid) | | |
| D | Citric Acid (10% in water) | q.s. to | q.s. to |
| | | pH 5.2 | pH 5.2 |

### Alcoholic Sun Care Spray SPF 30

| | | % | % |
|---|---|---|---|
| Phase | Raw material | w/w | w/w |
| A | TEGOSOFT ® CT (Caprylic/Capric | 20.00 | 20.00 |
| | Triglyceride) | | |
| | TEGOSOFT ® TN (C12-15 Alkyl Benzoate) | 8.00 | 8.00 |
| | TEGOSOFT ® APM (PPG-3 Myristyl Ether) | 8.00 | 8.00 |
| | Example X | 3.00 | 1.80 |
| | VP/Hexadecene Copolymer (Antaron V-216, | — | 0.70 |
| | Ashland) | | |
| | TEGO ® Xymenynic (Caprylic/Capric | 2.00 | 2.00 |
| | Triglyceride; Xymenynic Acid) | | |
| | Octocrylene | 6.80 | 6.80 |
| | Ethylhexyl Salicylate | 2.50 | 2.50 |
| | Butyl Methoxydibenzoylmethane | 5.00 | 5.00 |
| | Bis-Ethylhexyloxyphenol Methoxyphenyl | 3.00 | 3.00 |
| | Triazine (Tinosorb S, BASF SE) | | |
| | Diethylhexyl Butamido Triazone | 1.20 | 1.20 |
| B | Alcohol | to 100 | to 100 |

### Alcoholic Sun Care Spray SPF 30

| | | % | % |
|---|---|---|---|
| Phase | Raw material | w/w | w/w |
| A | TEGOSOFT ® CT (Caprylic/Capric | 20.00 | 20.00 |
| | Triglyceride) | | |
| | dermofeel ® sensolv MB (Isoamyl Laurate) | 8.00 | 8.00 |
| | TEGOSOFT ® APM (PPG-3 Myristyl Ether) | 8.00 | 8.00 |
| | Example X | 3.00 | 1.80 |
| | VP/Hexadecene Copolymer (Antaron V-216, | — | 0.70 |
| | Ashland) | | |
| | Tego ® Xymenynic (Caprylic/Capric | 2.00 | 2.00 |
| | Triglyceride; Xymenynic Acid) | | |
| | Octocrylene | 6.80 | 6.80 |
| | Ethylhexyl Salicylate | 2.50 | 2.50 |
| | Butyl Methoxydibenzoylmethane | 5.00 | 5.00 |
| | Bis-Ethylhexyloxyphenol Methoxyphenyl | 3.00 | 3.00 |
| | Triazine (Tinosorb S, BASF SE) | | |
| | Diethylhexyl Butamido Triazone | 1.20 | 1.20 |
| B | Alcohol | to 100 | to 100 |

### Transparent Sun Care Spray SPF 25

| Phase | Raw material | % w/w |
|---|---|---|
| A | TEGOSOFT ® P (Isopropyl Palmitate) | 12.00 |
| | Example X | 1.00 |
| | TEGOSOFT ® APM (PPG-3 Myristyl Ether) | 3.00 |
| | TEGOSOFT ® GC (PEG-7 Glyceryl Cocoate) | 1.50 |
| | TEGO ® Turmerone (Curcuma Longa (Turmeric) | 0.50 |
| | Root Extract) | |
| | Prunus Amygdalus Dulcis (Sweet Almond) Oil | 2.00 |
| | Dimethicone | 5.00 |
| | Homosalate | 10.00 |
| | Octocrylene | 9.40 |
| | Ethylhexyl Salicylate | 4.50 |
| | Butyl Methoxydibenzoylmethane | 3.00 |
| | Tocopheryl Acetate | 0.50 |
| B | Denat. Alcohol | to 100 |

### Water Resistant Aerosol Spray SPF 30

| | | % | % |
|---|---|---|---|
| Phase | Raw material | w/w | w/w |
| A | Denat. Alcohol | to 100 | to 100 |
| | Polyester-10 (and) Propylene Glycol | 1.50 | 1.50 |
| | Dibenzoate | | |
| | Example X | 0.75 | 3.00 |
| | TEGO ® SP 13 Sun Up MB (Poly C10-30 | 0.90 | — |
| | Alkyl Acrylate) | | |
| | Benzophenone-3 | 5.00 | 5.00 |
| | Ethylhexyl Salicylate | 5.00 | 5.00 |
| | Ethylhexyl Methoxycinnamate | 7.50 | 7.50 |
| | Homosalate | 10.00 | 10.00 |
| | Butyl Methoxydibenzoylmethane | 2.00 | 2.00 |
| | TEGOSOFT ® M (Isopropyl Myristate) | 4.00 | 4.00 |
| B | Drivosol ® 27D60 (Butane; Propane; | 10.00 | 20.00 |
| | Isobutane) | | |

### Water Resistant Aerosol Spray SPF 30

| | | % | % |
|---|---|---|---|
| Phase | Raw material | w/w | w/w |
| A | Denat. Alcohol | to 100 | to 100 |
| | Polyester-10 (and) Propylene Glycol | 1.50 | 1.50 |
| | Dibenzoate | | |
| | Example X | 0.75 | 3.00 |
| | TEGO ® SP 13 Sun Up MB (Poly C10-30 | 0.90 | — |
| | Alkyl Acrylate) | | |
| | Benzophenone-3 | 5.00 | 5.00 |
| | Ethylhexyl Salicylate | 5.00 | 5.00 |
| | Ethylhexyl Methoxycinnamate | 7.50 | 7.50 |
| | Homosalate | 10.00 | 10.00 |
| | Butyl Methoxydibenzoylmethane | 2.00 | 2.00 |
| | dermofeel ® sensolv MB (Isoamyl Laurate) | 6.00 | 6.00 |
| B | Drivosol ® 27D60 (Butane; Propane; | 10.00 | 20.00 |
| | Isobutane) | | |

### Light O/W Sun Care Lotion

| Phase | Raw material | % w/w | % w/w | % w/w | % w/w |
|---|---|---|---|---|---|
| A | AXOL ® C 62 Pellets MB (Glyceryl Stearate | 2.50 | 2.50 | 0.50 | 2.50 |
| | Citrate) | | | | |
| | Glyceryl Stearate SE | — | 1.00 | 4.00 | 1.00 |
| | Cetearyl Alcohol | 1.00 | 1.00 | — | 1.00 |
| | Glyceryl Stearate | — | — | 1.20 | — |
| | TEGOSOFT ® CT (Caprylic/Capric | 8.00 | 2.00 | — | 2.00 |
| | Triglyceride) | | | | |
| | TEGOSOFT ® TN (C12-15 Alkyl Benzoate) | — | 6.00 | 7.00 | 6.00 |
| | Diisopropyl Adipate | 1.00 | 1.00 | 1.00 | 1.00 |
| | Example X | 2.00 | 2.00 | 2.00 | 1.50 |
| | Butyloctyl Salicylate (HallBrite BHB, The | 2.00 | 2.00 | 2.00 | 2.00 |
| | HallStar Company) | | | | |
| | Tocopheryl Acetate | 0.20 | 0.20 | 0.20 | 0.20 |
| | Dimethicone (5 mPas) | 1.00 | — | — | 0.50 |
| | Tapioca Starch | — | 1.00 | — | — |
| | Hydroxyethyl Acrylate/Sodium | | | 1.20 | — |
| | Acryloyldimethyl Taurate Copolymer | | | | |
| | Triacontanyl PVP (Antaron WP-660, | — | — | — | 1.00 |
| | Ashland) | | | | |
| | Butyl Methoxydibenzoylmethane | 1.50 | 1.50 | 1.50 | 1.50 |
| | Diethylamino Hydroxybenzoyl Hexyl | 4.00 | 4.00 | 4.00 | 4.00 |
| | Benzoate (Uvinul A Plus, BASF) | | | | |
| | Ethylhexyl Triazone | 2.00 | 2.00 | 2.00 | 2.00 |
| | Ethylhexyl Salicylate | 5.00 | 5.00 | 5.00 | 5.00 |
| | Octocrylene | 8.00 | 8.00 | 8.00 | 8.00 |
| | Ethylhexyl Methoxycrylene | 1.50 | 1.50 | 1.50 | 1.50 |
| B | Titanium Dioxide; Trimethoxycaprylylsilane | 1.00 | 1.00 | 1.00 | 1.00 |
| C | Glycerin | 3.20 | 3.20 | 3.20 | 3.20 |
| | Propanediol | 2.50 | 2.50 | 2.50 | 2.50 |
| | EDTA | 0.02 | 0.02 | 0.02 | 0.02 |
| | Water | to 100 | to 100 | to 100 | to 100 |
| D | Acrylates/C10-30 Alkyl Acrylate | 0.10 | 0.10 | 0.10 | 0.10 |
| | Crosspolymer | | | | |
| | TEGOSOFT ® CT (Caprylic/Capric | 0.90 | 0.90 | 0.90 | 0.90 |
| | Triglyceride) | | | | |
| E | Sodium Hydroxide (10% in water) | q.s. | q.s. | q.s. | q.s. |
| Z | Preservative, Perfume | q.s. | q.s. | q.s. | q.s. |

### Dry Touch Hand Moisturizing Cream

| Phase | Raw material | % w/w | % w/w | % w/w |
|---|---|---|---|---|
| A | Varisoft ® TA 100 (Distearyldimonium Chloride) | 3.00 | 3.00 | — |
| | TEGO ® Care Eco CAT MB (Distearoylisopropyl | — | — | 4.50 |
| | Dimonium Methosulfate; Stearyl Alcohol) | | | |
| | Stearyl Alcohol | 1.00 | 1.00 | 1.00 |
| | TEGIN ® M Pellets MB (Glyceryl Stearate) | 1.00 | 1.00 | 2.00 |
| | TEGOSOFT ® SH (Stearyl Heptanoate) | 1.00 | 1.00 | 1.00 |
| | TEGOSOFT ® P (Isopropyl Palmitate) | 5.00 | 5.00 | 5.00 |
| | Example X | 1.50 | 2.00 | 2.00 |
| | Triacontanyl PVP (Antaron WP-660, Ashland) | 0.50 | — | — |
| | Diethylamino Hydroxybenzoyl Hexyl Benzoate (Uvinul A | 3.00 | 3.00 | 3.00 |
| | plus, BASF) | | | |
| | Ethylhexyl Salicylate | 3.00 | 3.00 | 3.00 |
| | Ethylhexyl Methoxycinnamate | 5.00 | 5.00 | 5.00 |
| | Tocopheryl Acetate | 0.50 | 0.50 | 0.50 |
| | TEGO ® Feel C 10 (Cellulose) | 1.00 | — | 1.00 |
| B | Water | to 100 | to 100 | to 100 |
| | Glycerin | 3.00 | 3.00 | 3.00 |
| C | TEGO ® Natural Betaine (Betaine) | 2.00 | 2.00 | 2.00 |
| | Water | 2.00 | 2.00 | 2.00 |
| D | Phenoxyethanol, Ethylhexylglycerin (Euxyl PE 9010, | 0.70 | 0.70 | 0.70 |
| | Schülke & Mayr GmbH) | | | |

### Age Defense BB Cream SPF 15

| Phase | Raw material | % w/w | % w/w | % w/w |
|---|---|---|---|---|
| A | C14-22 Alcohols; C12-20 Alkyl Glucoside | 4.50 | — | 3.50 |
| | Cetearyl Alcohol; Cetearyl Glucoside | — | 5.20 | — |
| | Potassium Cetyl Phosphate | 0.80 | — | — |
| | Sodium Stearoyl Glutamate | — | 1.10 | 2.00 |
| | Glyceryl Stearate | 1.00 | 1.00 | 1.00 |
| | Stearic Acid | 0.50 | 0.50 | 0.50 |
| | Stearyl Alcohol | 1.00 | 1.00 | 1.00 |
| | Butylene Glycol Dicaprylate/Dicaprate | 5.00 | 2.00 | 5.50 |
| | TEGOSOFT ® DEC (Diethylhexyl Carbonate) | 1.90 | 4.90 | 1.40 |
| | Capryloyl Glycerin/Sebacic Acid Copolymer | 1.50 | — | — |
| | (LexFilm Sun Natural MB, Inolex Inc.) | | | |
| | Example X | 1.50 | 3.00 | 3.00 |
| | HyaCare ® Filler CL (Water; Ethylhexyl Stearate; | 2.00 | 2.00 | 2.00 |
| | Sodium Hyaluronate Crosspolymer; Polyglyceryl-4 | | | |
| | Diisostearate/ Polyhydroxystearate/ Sebacate; Sodium | | | |
| | Isostearate) | | | |
| | Phytosphingosine | 0.10 | 0.10 | 0.10 |
| | Ethylhexyl Methoxycinnamate | 5.00 | 5.00 | 5.00 |
| | Diethylamino Hydroxybenzoyl Hexyl Benzoate | 3.00 | 3.00 | 3.00 |
| B | Titanium Dioxide (Hombitan AC 360, Sachtleben) | 3.00 | 3.00 | 3.00 |
| | Talc (Micro Talc IT Extra-AW, Mondo Minerals B.V.) | 2.00 | 2.00 | 2.00 |
| | Unipure Yellow LC 182 | 0.36 | 0.36 | 0.36 |
| | Unipure Red LC 381 | 0.12 | 0.12 | 0.12 |
| | Unipure Red LC 989 | 0.08 | 0.08 | 0.08 |
| | TEGOSOFT ® AC MB (Isoamyl Cocoate) | 4.40 | 4.40 | 4.40 |
| | TEGOSOFT ® CT (Caprylic/Capric Triglyceride) | 4.00 | 4.00 | 4.00 |
| | Polymethyl Methacrylate (Sepimat P, Seppic) | 3.00 | 3.00 | 3.00 |
| | Polyhydroxystearic Acid | 0.50 | 0.50 | — |
| C | Water | to 100 | to 100 | to 100 |
| | Glycerin | 3.00 | 3.00 | 3.00 |
| | HyaCare ® 50 (Hydrolyzed Hyaluronic Acid) | 0.10 | 0.10 | 0.10 |
| | TEGO ® Pep 4-17 (Tetrapeptide-21; Glycerin; Butylene | 2.00 | 2.00 | 2.00 |
| | Glycol; Water) | | | |
| D | Alcohol | 3.00 | 3.00 | 3.00 |
| E | Preservative | q.s. | q.s. | q.s. |

### Moisture Caring BB Cream SPF 15

| | | % | % |
|---|---|---|---|
| Phase | Raw material | w/w | w/w |
| A | ABIL ® EM 180 (Cetyl PEG/PPG-10/1 | 2.00 | 2.00 |
| | Dimethicone) | | |
| | ABIL ® Wax 9801 (Cetyl Dimethicone) | 2.00 | 2.00 |
| | Diethylamino Hydroxybenzoyl Hexyl | 10.00 | 10.00 |
| | Benzoate; Ethylhexyl Methoxycinnamate | | |
| | Dicaprylyl Carbonate | 2.00 | 2.00 |
| | Example X | 0.50 | 1.50 |
| | Dimethicone (and) Trimethylsiloxysilicate | 3.00 | 3.00 |
| | HyaCare ® Filler CL (Water; Ethylhexyl | 2.00 | 2.00 |
| | Stearate; Sodium Hyaluronate | | |
| | Crosspolymer; Polyglyceryl-4 | | |
| | Diisostearate/Polyhydroxystearate/ | | |
| | Sebacate; Sodium Isostearate) | | |
| | Phytosphingosine (Phytosphingosine) | 0.10 | 0.10 |
| | Hydrogenated Castor Oil | 0.50 | 0.50 |
| | Microcrystalline Wax | 0.50 | 0.50 |
| B | Talc (Micro Talc IT Extra-AW, Mondo | 2.00 | 2.00 |
| | Minerals B.V.) | | |
| | Titanium Dioxide (Hombitan AC 360, | 4.00 | 4.00 |
| | Sachtleben) | | |
| | Unipure Yellow LC 182 | 0.36 | 0.36 |
| | Unipure Red LC 381 | 0.12 | 0.12 |
| | Unipure Red LC 989 | 0.08 | 0.08 |
| | AEROSIL ® R 972 (Silica Dimethylsilylate) | 0.50 | — |
| | TEGOSOFT ® OS (Ethylhexyl Stearate) | 5.00 | 5.00 |
| | TEGOSOFT ® DEC (Diethylhexyl Carbonate) | 6.00 | 6.00 |
| C | Water | to 100 | to 100 |
| | HyaCare ® (Sodium Hyaluronate) | 0.05 | 0.05 |
| | Glycerin | 5.00 | 5.00 |
| | Sodium Chloride | 0.80 | 0.80 |
| D | Phenoxyethanol; Ethylhexylglycerin (Euxyl | 0.70 | 0.70 |
| | PE 9010, Schülke & Mayr) | | |

### Anhydrous Stick

| Phase | Raw material | % w/w |
|---|---|---|
| | dermofeel ® viscolid MB (Hydrogenated Vegetable Oil) | 10.00 |
| | TEGO ® SP 13 Sun Up (Poly C10-30 Alkyl Acrylate) | 5.00 |
| | Example X | 10.00 |
| | TEGOSOFT ® CT (Caprylic/Capric Triglyceride) | 30.00 |
| | Isopropyl Palmitate | 15.00 |
| | Ethylhexyl Methoxycinnamate | 7.50 |
| | Octyldodecanol | to 100 |

### O/W Sun Protect & Bronze

| Phase | Raw material | % w/w | % w/w | % w/w | % w/w |
|---|---|---|---|---|---|
| A | Glyceryl Stearate Citrate | 2.00 | 2.00 | 2.00 | 2.00 |
| | TEGO ® Alkanol 1618 (Cetearyl Alcohol) | 1.50 | 1.50 | 1.50 | 1.50 |
| | TEGOSOFT ® DC MB (Decyl Cocoate) | 8.00 | 8.00 | 8.00 | 8.00 |
| | TEGOSOFT ® XC MB (Phenoxyethyl | 4.00 | 4.00 | 4.00 | 4.00 |
| | Caprylate) | | | | |
| | Cera Alba (Beeswax 8104, KahlWax) | 0.50 | — | — | — |
| | Example X | 0.50 | 2.00 | 1.10 | 1.30 |
| | Styrene/Acrylates Copolymer (Dermacryl E, | — | — | 1.00 | — |
| | Nouryon) | | | | |
| | Acrylates/Octylacrylamide Copolymer | — | — | — | 0.8 |
| | (Dermacryl 79, Nouryon) | | | | |
| | Titanium Dioxide, Trimethoxycaprylylsilane | 1.50 | 1.50 | 1.50 | 1.50 |
| | Dimethicone (5 cSt) | 3.00 | — | — | — |
| | Talc | — | 1.20 | 1.20 | 1.20 |
| | Octocrylene | 9.00 | 9.00 | 9.00 | 9.00 |
| | Ethylhexyl Salicylate | 4.50 | 4.50 | 4.50 | 4.50 |
| | Butyl Methoxydibenzoylmethane | 2.00 | 2.00 | 2.00 | 2.00 |
| | Tocopheryl Acetate | 0.50 | 0.50 | 0.50 | 0.50 |
| | Xanthan Gum (Keltrol CG-SFT, CP Kelco) | 0.50 | 0.50 | 0.50 | 0.50 |
| B | Water | to 100 | to 100 | to 100 | to 100 |
| | Glycerin | 3.00 | 3.00 | 3.00 | 3.00 |
| | Panthenol | 1.00 | 1.00 | 1.00 | 1.00 |
| C | Acrylates/C10-30 Alkyl Acrylate | 0.10 | 0.10 | 0.10 | 0.10 |
| | Crosspolymer | | | | |
| | TEGOSOFT ® TN (C12-15 Alkyl Benzoate) | 0.40 | 0.40 | 0.40 | 0.40 |
| D | Sodium Hydroxide (10% in water) | q.s. | q.s. | q.s. | q.s. |
| E | Butylene Glycol; Acetyl-L-Tyrosine; | 0.80 | 0.80 | 0.80 | 0.80 |
| | Hydrolyzed Vegetable Protein; Adenosine | | | | |
| | Triphosphate; Riboflavin (Unipertan VEG- | | | | |
| | 2001) | | | | |
| Z | Preservative, Perfume | q.s. | q.s. | q.s. | q.s. |

| Phase | Raw material | % w/w | % w/w | % w/w |
|---|---|---|---|---|
| A | TEGO ® Care PBS 6 MB (Polyglyceryl-6 Stearate, | 2.00 | 2.00 | 2.00 |
| | Polyglyceryl-6 Behenate) | | | |
| | TEGOSOFT ® AC MB (Isoamyl Cocoate) | 5.00 | 5.00 | 5.00 |
| | dermofeel ® viscolid MB (Hydrogenated Vegetable Oil) | 0.50 | 0.50 | 0.50 |
| | Example X | 0.50 | 0.50 | 0.50 |
| | Bis-Ethylhexyloxyphenol Methoxyphenyl Triazine | 4.50 | 4.50 | 4.50 |
| | Butyl Methoxydibenzoylmethane | 3.00 | 3.00 | 3.00 |
| | Octocrylene | 8.00 | 8.00 | 8.00 |
| | Homomenthyl Salicylate | 6.00 | 6.00 | 6.00 |
| | Ethylhexyl Salicylate | 2.50 | 2.50 | 2.50 |
| | TEGO ® Feel C 10 (Cellulose) | 2.00 | — | — |
| | Methyl Methacrylate Crosspolymer | — | 1.50 | — |
| | Aluminum Starch Octenylsuccinate | — | — | 1.70 |
| B | Water | to 100 | to 100 | to 100 |
| | Glycerin | 2.00 | 2.00 | 2.00 |
| | EDTA Disodium | 0.05 | 0.05 | 0.05 |
| C | TEGOSOFT ® TN (C12-15 Alkyl Benzoate) | 0.80 | 0.80 | 0.80 |
| | Carbomer | 0.20 | 0.20 | 0.20 |
| | Xanthan Gum (Keltrol CG-SFT, CP Kelco) | 0.20 | 0.20 | 0.20 |
| D | Water | 11.68 | 11.68 | 11.68 |
| | Phenylbenzimidazole Sulfonic Acid | 3.00 | 3.00 | 3.00 |
| | Tromethamine | 1.32 | 1.32 | 1.32 |
| E | Phenoxyethanol, Ethylhexylglycerin (Euxyl PE 9010, | 0.70 | 0.70 | 0.70 |
| | Schülke & Mayr GmbH) | | | |
| | TEGO ® Betain 810 (Capryl/Capramidopropyl Betaine) | 1.00 | 1.00 | 1.00 |
| | REWOTERIC ® AM C (Sodium Cocoamphoacetate) | 1.00 | 1.00 | 1.00 |
| F | Tromethamine (30% in water) | q.s. | q.s. | q.s. |

### W/O Sun Protection Shake-Shake

| Phase | Raw material | % w/w |
|---|---|---|
| A | ABIL ® EM 90 (Cetyl PEG/PPG-10/1 Dimethicone) | 1.00 |
| | TEGOSOFT ® CT (Caprylic/Capric Triglyceride) | 11.00 |
| | Dicaprylyl Carbonate | 2.50 |
| | Dimethicone (5 cSt) | 12.00 |
| | Octocrylene | 6.00 |
| | Ethylhexyl Salicylate | 2.00 |
| | Butyl Methoxydibenzoylmethane | 0.80 |
| | Bis-Ethylhexyloxyphenol Methoxyphenyl Triazine | 1.50 |
| | (Tinosorb S, BASF SE) | |
| | Titanium Dioxide, Trimethoxycaprylylsilane | 2.00 |
| | Example X | 0.50 |
| B | Water | 4.43 |
| | Phenylbenzimidazole Sulfonic Acid (Eusolex 232, | 0.50 |
| | Merck KGaA) | |
| | Sodium Hydroxide | 0.07 |
| C | Distarch Phosphate (MAIS PO4 PH “B”, Agrana | 1.50 |
| | Stärke GmbH) | |
| | Alcohol | 1.00 |
| | Glycerin | 6.00 |
| | Water | to 100 |
| Z | Preservative, Perfume | q.s. |

### Light Sun Care W/O Shake-Shake SPF30 PA+++

| Phase | Raw material | % w/w |
|---|---|---|
| A | ABIL ® EM 90 (Cetyl PEG/PPG-10/1 Dimethicone) | 1.50 |
| | TEGOSOFT ® DEC (Diethylhexyl Carbonate) | 5.00 |
| | Dimethicone (2 cSt) | 20.00 |
| | TEGOSOFT ® CT (Caprylic/Capric Triglyceride) | 5.00 |
| | C12-15 Alkyl Benzoate; Titanium Dioxide; | 2.40 |
| | PEG-30 Dipolyhydroxystearate; | |
| | Silica; Dimethicone (T-lique AB50Si, Uni-Powder) | |
| | Zinc Oxide (ZnO-NAS, Sunjin) | 8.00 |
| | ABIL ® Wax 9801 (Cetyl Dimethicone) | 2.00 |
| | Talc | 2.00 |
| | Polymethylsilsesquioxane (Gransil PSQ, Grant) | 3.00 |
| | Vinyl Dimethicone/Methicone Silsesquioxane | 1.00 |
| | Crosspolymer (KSP-100, ShinEtsu) | |
| | Example X | 2.00 |
| | Diethylamino Hydroxybenzoyl Hexyl Benzoate | 2.00 |
| | Ethylhexyl Methoxycinnamate | 8.00 |
| | Ethylhexyl Salicylate | 4.00 |
| | Bis-Ethylhexyloxyphenol Methoxyphenyl Triazine | 0.50 |
| | (Escalol S, Ashland) | |
| B | Water | to 100 |
| | Glycerin | 3.00 |
| | Sodium Chloride | 1.00 |
| C | Phenoxyethanol, Ethylhexylglycerin (Euxyl | 0.70 |
| | PE 9010, Schülke & Mayr GmbH) | |

W/O Organic shake-shake SPF50+PA++++

| Phase | Raw material | % w/w |
|---|---|---|
| A | ABIL ® EM 90 (Cetyl PEG/PPG-10/1 Dimethicone) | 1.50 |
| | TEGOSOFT ® DEC (Diethylhexyl Carbonate) | 7.00 |
| | Dimethicone (5 cSt) | 5.00 |
| | Cyclopentasiloxane; Cyclohexasiloxane | 5.00 |
| | TEGOSOFT ® P (Isopropyl Palmitate) | 3.00 |
| | Example X | 0.50 |
| | C12-15 Alkyl Benzoate; Titanium Dioxide; | 15.60 |
| | PEG-30 Dipolyhydroxystearate; | |
| | Silica; Dimethicone (T-lique AB50Si, Uni-Powder) | |
| | ABIL ® Wax 9801 (Cetyl Dimethicone) | 2.00 |
| | Bis-Ethylhexyloxyphenol Methoxyphenyl Triazine | 1.00 |
| | (Escalol S, Ashland) | |
| | Diethylamino Hydroxybenzoyl Hexyl Benzoate | 1.50 |
| | (Uvinul A plus Granular, BASF) | |
| | Octocrylene | 2.00 |
| | Ethylhexyl Methoxycinnamate | 7.00 |
| | Ethylhexyl Salicylate | 4.00 |
| | Zinc Oxide (ZnO-NAS, Sunjin) | 14.40 |
| B | Water | to 100 |
| | Butylene Glycol | 3.00 |
| | Sodium Chloride | 1.50 |
| C | Phenoxyethanol, Ethylhexylglycerin (Euxyl | 0.70 |
| | PE 9010, Schülke & Mayr GmbH) | |

### Sun Care Cream SPF 30

| | | % | % |
|---|---|---|---|
| Phase | Raw material | w/w | w/w |
| A | dermofeel ® GSC SG (Glyceryl Stearate | 2.00 | 2.00 |
| | Citrate) | | |
| | TEGIN ® M Pellets MB (Glyceryl Stearate) | 0.50 | 0.50 |
| | TEGO ® Alkanol 1618 (Cetearyl Alcohol) | 0.50 | 0.50 |
| | dermofeel ® sensolv MB (Isoamyl Laurate) | 4.50 | 4.50 |
| | Bis-Ethylhexyloxyphenol Methoxyphenyl | 3.00 | 3.00 |
| | Triazine (Tinosorb S, BASF) | | |
| | Butyl Methoxydibenzoylmethane | 2.00 | 2.00 |
| | Ethylhexyl Salicylate | 3.00 | 3.00 |
| | Octocrylene | 8.00 | 8.00 |
| | Homosalate | 3.00 | 3.00 |
| | Ethylhexyl Triazone | 1.50 | 1.50 |
| | Example X | 1.50 | 1.50 |
| B | Water | to 100 | to 100 |
| | Glycerin | 3.00 | 3.00 |
| | EDTA | 0.05 | 0.05 |
| | TEGO ® Feel C 10 (Cellulose) | 2.00 | — |
| | Zea Mays (Corn) Starch | — | 2.00 |
| | 1,2-Hexanediol | 2.20 | 2.20 |
| C | Acrylates/C10-30 Alkyl Acrylate Crosspolymer | 0.20 | 0.20 |
| | dermofeel ® sensolv MB (Isoamyl Laurate) | 0.80 | 0.80 |
| D | Sodium Hydroxide (10% in water) | q.s. | q.s. |
| E | Verstatil ® PC (Phenoxyethanol, Caprylyl | 1.00 | 1.00 |
| | Glycol) | | |

### On the go UV Protection Stick SPF 50

| | | % |
|---|---|---|
| Phase | Raw material | w/w |
| A | TEGOSOFT ® M (Isopropyl Myristate) | 8.00 |
| | TEGOSOFT ® CT (Caprylic/Capric Triglyceride) | 7.50 |
| | TEGOSOFT ® DEC (Diethylhexyl Carbonate) | 8.60 |
| | Titanium Dioxide, Silica, Dimethicone | 2.50 |
| | (Parsol TX, DSM) | |
| B | TEGOSOFT ® CR MB (Cetyl Ricinoleate) | 2.60 |
| | TEGOSOFT ® MM MB (Myristyl Myristate) | 4.00 |
| | Diethylamino Hydroxybenzoyl Hexyl Benzoate | 8.00 |
| | Ethylhexyl Salicylate | 5.00 |
| | Ethylhexyl Methoxycinnamate | 10.00 |
| | Homosalate | 5.00 |
| | Bis-Ethylhexyloxyphenol Methoxyphenyl Triazine | 3.50 |
| | (Tinosorb S, BASF) | |
| | Beeswax | 3.50 |
| | Polyglyceryl-3 Beeswax (Cera Bellina, Koster | 6.00 |
| | Keunen) | |
| | Ozokerite (1899, KahlWax) | 3.50 |
| | Copernicia Cerifera (Carnauba) Wax (2442L, | 2.50 |
| | KahlWax) | |
| | dermofeel ® AP MB (Ascorbyl Palmitate) | 0.10 |
| | dermofeel ® Toco 70 non GMO (Tocopherol, | 0.20 |
| | Helianthus Annuus (Sunflower) Seed Oil) | |
| | TEGO ® SP 13-1 (Poly C10-30 Alkyl Acrylate) | 2.50 |
| | Example X | 5.00 |
| C | AEROSIL ® 200 (Silica) | 2.00 |
| D | TEGO ® Feel C 10 (Cellulose) | 10.00 |
| Z | Perfume | q.s. |

### Transparent UV Protection Water Spray SPF 30

| | | % |
|---|---|---|
| Phase | Raw material | w/w |
| A | TEGOSOFT ® DEC (Diethylhexyl Carbonate) | 15.00 |
| | Isododecane | 7.50 |
| | TEGOSOFT ® P (Isopropyl Palmitate) | 5.00 |
| | dermofeel ® sensolv MB (Isoamyl Laurate) | 5.00 |
| | Butyl Methoxydibenzoylmethane | 5.00 |
| | Bis-Ethylhexyloxyphenol Methoxyphenyl Triazine | 2.40 |
| | (Tinosorb S, BASF) | |
| | Homosalate | 2.00 |
| | Ethylhexyl Salicylate | 2.00 |
| | Ethylhexyl Triazone | 1.70 |
| | dermofeel ® Toco 70 non GMO (Tocopherol, | 0.50 |
| | Helianthus Annuus (Sunflower) Seed Oil) | |
| | Example X | 0.35 |
| B | Verstatil ® PC (Phenoxyethanol, Caprylyl Glycol) | 1.00 |
| | Perfume | q.s. |
| C | Water | to 100 |
| | Glycerin | 3.00 |
| | Disodium EDTA | 0.10 |
| D | Water | 9.65 |
| | Phenylbenzimidazole Sulfonic Acid | 2.50 |
| | Sodium Hydroxide | 0.35 |
| E | Triethanolamine | q.s. to |
| | | pH 7.1 |

### Sun Care Cream SPF 25

| | | % | % |
|---|---|---|---|
| Phase | Raw material | w/w | w/w |
| A | Glyceryl Stearate | 0.50 | 0.50 |
| | Cetearyl Alcohol | 0.50 | — |
| | Cetyl Alcohol | — | 0.80 |
| | dermofeel ® sensolv MB (Isoamyl Laurate) | 3.50 | 3.50 |
| | Butyl Methoxydibenzoylmethane | 3.00 | 3.00 |
| | Ethylhexyl Salicylate | 5.00 | 5.00 |
| | Octocrylene | 10.0 | 10.0 |
| | Homosalate | 10.0 | 10.0 |
| | Example X | 1.50 | 1.50 |
| B | Potassium Cetyl Phosphate (Amphisol K, | 1.50 | 1.50 |
| | DSM Nutritional Products) | | |
| | Water | to 100 | to 100 |
| | Glycerin | 3.00 | 3.00 |
| | EDTA | 0.05 | 0.05 |
| C | Acrylates/C10-30 Alkyl Acrylate Crosspolymer | 0.20 | 0.20 |
| | dermofeel ® sensolv MB (Isoamyl Laurate) | 0.80 | 0.80 |
| D | Sodium Hydroxide (10% in water) | 0.60 | 0.60 |
| E | Verstatil ® PC (Phenoxyethanol, Caprylyl | 1.00 | 1.00 |
| | Glycol) | | |

### Inorganic Water-Resistant O/W Sunscreen SPF 20

| Phase | Raw material | % w/w | % w/w |
|---|---|---|---|
| A | TEGO ® Care PBS 6 MB (Polyglyceryl-6 | 4.00 | 4.00 |
| | Stearate; Polyglyceryl-6 Behenate) | | |
| | Tego ® SP 13-1 (Poly C10-30 Alkyl Acrylate) | 1.50 | — |
| | TEGIN ® M Pellets MB (Glyceryl Stearate) | 0.20 | 0.20 |
| | TEGO ® Alkanol 1618 (Cetearyl Alcohol) | 0.20 | 0.20 |
| B | Zinc Oxide; Triethoxycaprylylsilane | 7.80 | 7.80 |
| | (Z-Cote HP-1, BASF) | | |
| | Titanium Dioxide, Silica, Dimethicone | 7.20 | 7.20 |
| | (Parsol TX, DSM) | | |
| | TEGOSOFT ® DC MB (Decyl Cocoate) | 3.00 | 3.00 |
| | dermofeel ® sensolv MB (Isoamyl Laurate) | 3.00 | 3.00 |
| | TEGOSOFT ® CT (Caprylic/Capric | 5.00 | 5.00 |
| | Triglyceride) | | |
| | TEGOSOFT ® DEC (Diethylhexyl Carbonate) | 5.00 | 5.00 |
| | ABIL ® 350 (Dimethicone) | 3.00 | — |
| | ABIL ® Wax 9840 (Cetyl Dimethicone) | 2.00 | — |
| | Zea Mays (Corn) Starch | — | 2.00 |
| | dermosoft ® Octiol (Caprylyl Glycol) | 0.50 | 0.50 |
| | Example X | 0.50 | 2.50 |
| C | Water | to 100 | to 100 |
| | Glycerin | 3.00 | 3.00 |
| | Xanthan Gum (Keltrol CG-SFT, CP Kelco) | 0.50 | 0.50 |
| D | dermosoft ® Pentiol eco (Pentylene Glycol) | 3.00 | 3.00 |
| E | Citric Acid (50% in water) | 0.45 | 0.45 |

### Feel the Sun Spray SPF 50

| | | % | % |
|---|---|---|---|
| Phase | Raw material | w/w | w/w |
| A | dermofeel ® GSC SG (Glyceryl Stearate | 2.50 | 2.50 |
| | Citrate) | | |
| | TEGIN ® M Pellets MB (Glyceryl Stearate) | 0.25 | 0.25 |
| | TEGO ® Alkanol 1618 (Cetearyl Alcohol) | 0.25 | 0.25 |
| | dermofeel ® sensolv MB (Isoamyl Laurate) | 4.00 | 6.00 |
| | Di(iso)pentyl terephthalate | 4.00 | — |
| | Example X | 1.00 | 1.00 |
| | Bis-Ethylhexyloxyphenol Methoxyphenyl | 6.00 | 6.00 |
| | Triazine (Tinosorb S, BASF) | | |
| | Diethylamino Hydroxybenzoyl Hexyl Benzoate | 6.00 | 6.00 |
| | Ethylhexyl Salicylate | 5.00 | 5.00 |
| | Ethylhexyl Triazone | 2.00 | 2.00 |
| | dermofeel ® Toco 70 non GMO (Tocopherol, | 0.20 | 0.20 |
| | Helianthus Annuus (Sunflower) Seed Oil) | | |
| | Titanium Dioxide; Aluminum Hydroxide; | — | 1.50 |
| | Stearic Acid (A15-TiO₂-ST-SA8, | | |
| | Kobo Products) | | |
| B | Water | to 100 | to 100 |
| | Glycerin | 3.00 | 3.00 |
| | Propylene Glycol | 2.00 | 2.00 |
| | EDTA | 0.05 | 0.05 |
| C | Xanthan Gum (Keltrol CG-SFT, CP Kelco) | 0.50 | 0.50 |
| D | Phenylbenzimidazole Sulfonic Acid | 3.00 | 3.00 |
| | Tromethamine (Trisaminomethane) | 1.32 | 1.32 |
| | Water | 10.68 | 10.68 |
| E | Verstatil ® BOB (Benzyl Alcohol; Caprylyl | 1.20 | 1.20 |
| | Glycol; Benzoic Acid) | | |
| Z | Perfume | 0.10 | 0.10 |

### Natural Sun Protection Stick

| Phase | Raw material | % w/w |
|---|---|---|
| A | Carthamus Tinctorius (Safflower) Seed Oil | 22.80 |
| | Butyrospermum Parkii Butter (shea butter) | 7.00 |
| | Hippophae Rhamnoides Fruit Oil | 1.00 |
| | dermofeel ® sensolv MB (Isoamyl Laurate) | 8.00 |
| | TEGOSOFT ® CT (Caprylic/Capric Triglyceride) | 2.00 |
| | dermofeel ® viscolid MB (Hydrogenated Vegetable Oil) | 2.00 |
| | TEGOSOFT ® DC MB (Decyl Cocoate) | 6.00 |
| | TEGOSOFT ® OER MB (Oleyl Erucate) | 6.00 |
| | Cera Alba (Beeswax 8104, KahlWax) | 5.00 |
| | Helianthus Annuus Seed Cera, Ascorbyl Palmitate, Tocopherol | 3.00 |
| | (Sunflower Seed Wax 6607L, KahlWax) | |
| | Euphorbia Cerifera Cera (Candelilla Wax 2039L, KahlWax) | 3.00 |
| | Example X | 8.00 |
| | dermofeel ® TocoBalance | 1.50 |
| | (Tocopherol, Helianthus Annuus (Sunflower) Seed Oil) | |
| | dermosoft ® GMCY MB (Glyceryl Caprylate) | 1.00 |
| B | Distarch Phosphate (Corn P04 PH “B”, Agrana) | 1.00 |
| | TEGO ® Feel C 10 (Cellulose) | 2.00 |
| | Zinc Oxide (Zano 10, EverZinc) | 20.00 |
| C | Perfume | 0.70 |

### W/O Quick-Breaking Cream SPF 15

| Phase | Raw material | % w/w |
|---|---|---|
| A | ABIL ® EM 180 (Cetyl PEG/PPG-10/1 Dimethicone) | 0.80 |
| | TEGOSOFT ® DEC (Diethylhexyl Carbonate) | 3.00 |
| | TEGOSOFT ® CT (Caprylic/Capric Triglyceride) | 2.00 |
| | Dimethicone (5 cSt) | 3.00 |
| | Example X | 1.00 |
| | Bis-Ethylhexyloxyphenol Methoxyphenyl Triazine (Tinosorb S, BASF) | 2.00 |
| | Diethylamino Hydroxybenzoyl Hexyl Benzoate | 2.00 |
| | 4-Methylbenzylidene Camphor | 4.00 |
| | Ethylhexyl Salicylate | 2.00 |
| | Verstatil ® BOB (Benzyl Alcohol, Caprylyl Glycol, Benzoic Acid) | 1.00 |
| B | Water | to 100 |
| | Propylene Glycol | 5.00 |
| | Sodium Chloride | 1.00 |

### W/O Sun Care Lotion SPF 8, Water Resistant

| | | % | % | % | % |
|---|---|---|---|---|---|
| Phase | Raw material | w/w | w/w | w/w | w/w |
| A | ISOLAN ® GPS | 3.00 | — | — | — |
| | (Polyglyceryl-4 Diisostearate/Polyhydroxystearate/ | | | | |
| | Sebacate) | | | | |
| | Polyglyceryl-3 Diisostearate | — | 3.50 | — | — |
| | Polyglyceryl-2 Dipolyhydroxystearate | — | — | 3.00 | — |
| | Polyglyceryl-3 Polyricinoleate | — | — | — | 3.00 |
| | Hydrogenated Castor Oil | 0.20 | 0.20 | 0.20 | 0.20 |
| | Microcrystalline Wax | 0.30 | 0.30 | 0.30 | 0.30 |
| | TEGOSOFT ® CT (Caprylic/Capric Triglyceride) | 5.00 | 5.00 | 5.00 | 5.00 |
| | TEGOSOFT ® DEC (Diethylhexyl Carbonate) | 3.00 | 3.00 | 3.00 | 3.00 |
| | TEGOSOFT ® CR MB (Cetyl Ricinoleate) | 0.75 | 0.75 | 0.75 | 0.75 |
| | Example X | 0.75 | 0.75 | 0.75 | 0.75 |
| | Triisostearin | 1.00 | 1.00 | 1.00 | 1.00 |
| | Ethylhexyl Methoxycinnamate | 5.00 | 5.00 | 5.00 | 5.00 |
| | Ethylhexyl Salicylate | 4.00 | 4.00 | 4.00 | 4.00 |
| | Menthyl Anthranilate | 4.00 | 4.00 | 4.00 | 4.00 |
| B | Tego ® Cosmo C 100 (Creatine) | 0.50 | 0.50 | 0.50 | 0.50 |
| | Glycerin | 3.00 | 3.00 | 3.00 | 3.00 |
| | Magnesium Sulfate Heptahydrate | 1.50 | 1.50 | 1.50 | 1.50 |
| | Water | to 100 | to 100 | to 100 | to 100 |
| Z | Preservative, Perfume | q.s. | q.s. | q.s. | q.s. |

### W/O Sun Care Lotion, Water Resistant

| Phase | Raw material | % w/w |
|---|---|---|
| A | ISOLAN ® 17 MB (Polyglyceryl-4 Diisostearate/Polyhydroxystearate/ | 4.00 |
| | Sebacate; Caprylic/Capric Triglyceride; Polyglyceryl-3 Oleate; Diisostearoyl | |
| | Polyglyceryl-3 Dimer Dilinoleate) | |
| | Paraffin; Cera Microcristallina (Paracera W 80, Paramelt) | 0.25 |
| | Hydrogenated Castor Oil | 0.25 |
| | TEGOSOFT ® XC MB (Phenoxyethyl Caprylate) | 7.00 |
| | Bis-Ethylhexyloxyphenol Methoxyphenyl Triazine (Tinosorb S, BASF) | 7.00 |
| | Homosalate | 10.00 |
| | Diethylamino Hydroxybenzoyl Hexyl Benzoate | 6.00 |
| | Example X | 1.00 |
| | dermofeel ® Toco 70 non GMO | 0.50 |
| | (Tocopherol, Helianthus Annuus (Sunflower) Seed Oil) | |
| | dermosoft ® GMC MB (Glyceryl Caprate) | 0.50 |
| B | Water | to 100 |
| | Glycerin | 3.00 |
| | Zinc Sulfate Heptahydrate | 1.00 |

### Cationic Sun Screen SPF 19, Water Resistant

| Phase | Raw material | % w/w |
|---|---|---|
| A | Varisoft ® TA 100 (Distearyldimonium Chloride) | 3.50 |
| | TEGIN ® M Pellets MB (Glyceryl Stearate) | 2.00 |
| | Stearyl Alcohol | 1.00 |
| | TEGOSOFT ® CT (Caprylic/Capric Triglyceride) | 5.00 |
| | TEGOSOFT ® DEC (Diethylhexyl Carbonate) | 3.50 |
| | Example X | 1.50 |
| | Triisostearin | 1.00 |
| | Titanium Dioxide, Diethylhexyl Carbonate, Polyglyceryl-6 | 5.00 |
| | Polyhydroxystearate | |
| | 4-Methylbenzylidene Camphor | 4.00 |
| | Octocrylene | 3.00 |
| | Butyl Methoxydibenzoylmethane | 2.00 |
| B | Glycerin | 3.00 |
| | Water | to 100 |
| Z | Preservative, Perfume | q.s. |

### Cationic Sun Screen SPF 15, Water Resistant

| Phase | Raw material | % w/w |
|---|---|---|
| A | Varisoft ® TA 100 (Distearyldimonium Chloride) | 3.50 |
| | TEGIN ® M Pellets MB (Glyceryl Stearate) | 1.50 |
| | Stearyl Alcohol | 1.00 |
| | TEGOSOFT ® DC MB (Decyl Cocoate) | 8.00 |
| | Dicaprylyl Carbonate | 5.00 |
| | TEGOSOFT ® MM MB (Myristyl Myristate) | 1.00 |
| | Example X | 2.00 |
| | TEGOSOFT ® OER MB (Oleyl Erucate) | 1.00 |
| | Ethylhexyl Methoxycinnamate | 5.00 |
| | Bis-Ethylhexyloxyphenol Methoxyphenyl Triazine | 3.00 |
| B | TEGO ® Cosmo C 100 (Creatine) | 0.50 |
| | Glycerin | 3.00 |
| | Water | to 100 |
| Z | Preservative, Perfume | q.s. |

### Everyday Sunshine Cream SPF 15

| Phase | Raw material | % w/w | % w/w | % w/w |
|---|---|---|---|---|
| A | TEGO ® Care 165 (Glyceryl Stearate; PEG-100 | 3.00 | 3.00 | — |
| | Stearate) | | | |
| | Polysorbate 60 | — | — | 4.00 |
| | Stearyl Alcohol | 2.00 | 1.50 | 3.00 |
| | TEGOSOFT ® CT (Caprylic/Capric Triglyceride) | 4.00 | 4.00 | 4.00 |
| | Propylene Glycol Dibenzoate | 2.00 | 2.00 | 2.00 |
| | Titanium Dioxide; Trimethoxycaprylylsilane | 1.00 | 1.00 | 1.00 |
| | Paraffinum Liquidum | 2.00 | 2.00 | 2.00 |
| | Butyrospermum Parkii (Shea Butter) | 1.00 | 1.00 | 1.00 |
| | Octocrylene | 2.00 | 2.00 | 2.00 |
| | Butyl Methoxydibenzoylmethane | 2.00 | 2.00 | 2.00 |
| | Ethylhexyl Salicylate | 5.00 | 5.00 | 5.00 |
| | Example X | 1.50 | 1.50 | 1.50 |
| | Tocopheryl Acetate | 0.80 | 0.80 | 0.80 |
| B | Water | to 100 | to 100 | to 100 |
| | Glycerin | 3.00 | 3.00 | 3.00 |
| | Acrylates Copolymer | — | 1.00 | — |
| | Graham's salt (Sodium Hexametaphosphate) | 0.20 | 0.20 | 0.20 |
| | TEGO ® Cosmo C 100 (Creatine) | 0.50 | 0.50 | 0.50 |
| | HyaCare ® 50 (Hydrolyzed Hyaluronic Acid) | 0.10 | 0.10 | 0.10 |
| | Polyacrylamide; C13-14 Isoparaffin; Laureth-7 | 2.00 | 1.20 | 2.00 |
| | (Sepigel 305, Seppic) | | | |
| C | Water | 13.29 | 13.29 | 13.29 |
| | Phenylbenzimidazole Sulfonic Acid (Eusolex 232, | 1.05 | 1.05 | 1.05 |
| | Merck KGaA) | | | |
| | Sodium Hydroxide | q.s. | q.s. | q.s. |
| Z | Preservative, perfume | q.s. | q.s. | q.s. |

### High Sun Protection Lotion O/W SPF 50

| Phase | Raw material | % w/w | % w/w | % w/w |
|---|---|---|---|---|
| A | Polyglyceryl-3 Distearate; Glyceryl Stearate Citrate | 3.50 | 3.00 | 3.00 |
| | Sodium Cetearyl Sulfate | — | 0.50 | 0.50 |
| | TEGOSOFT ® CT (Caprylic/Capric Triglyceride) | 5.00 | 5.00 | 3.00 |
| | Example X | 0.50 | 0.50 | 0.50 |
| | REWOPAL ® PIB 1000 (Polyisobutene) | 0.50 | 0.50 | 0.50 |
| | Acrylates/C10-30 Alkyl Acrylate Crosspolymer | 0.20 | 0.20 | — |
| | Xanthan Gum (Keltrol CG-SFT, CP Kelco) | 0.20 | 0.20 | 0.05 |
| | Titanium Dioxide; Trimethoxycaprylylsilane | 1.50 | 1.50 | 1.50 |
| | Bis-Ethylhexyloxyphenol Methoxyphenyl Triazine | 4.00 | 4.00 | 4.00 |
| | (Tinosorb S, BASF SE) | | | |
| | Octocrylene | 10.00 | 10.00 | 10.00 |
| | Butyl Methoxydibenzoylmethane | 4.00 | 4.00 | 4.00 |
| B | Glycerin | 4.00 | to 100 | to 100 |
| | Water | to 100 | 5.00 | 5.00 |
| C | Alcohol | 5.00 | q.s. | q.s. |
| | TEGOSOFT ® CT (Caprylic/Capric Triglyceride) | — | — | 2.20 |
| | Disteardimonium Hectorite | — | — | 0.20 |
| | Propylene Carbonate | — | — | 0.20 |
| | Preservative | q.s. | q.s. | q.s. |
| D | Methylene Bis-Benzotriazolyl Tetramethylbutylphenol | 8.00 | q.s. | q.s. |
| | (Tinosorb M, BASF SE) | | | |
| E | Sodium Hydroxide (10% in water) | q.s. to | q.s. to | q.s. to |
| | | pH 6 | pH 6 | pH 6 |

### Icy O/W Sun Care Lotion SPF 25

| Phase | Raw material | % w/w | % w/w | % w/w | % w/w |
|---|---|---|---|---|---|
| A | Polyglyceryl-3 Distearate | 3.00 | 1.00 | — | — |
| | Sucrose Polystearate; Cetyl Palmitate | — | — | 2.50 | 3.20 |
| | Sodium Stearoyl Glutamate | — | 1.50 | 1.00 | — |
| | Stearyl Alcohol | 1.00 | 1.00 | 1.00 | 1.00 |
| | dermofeel ® sensolv MB (Isoamyl Laurate) | 5.00 | 5.00 | 5.00 | 5.00 |
| | TEGOSOFT ® SH (Stearyl Heptanoate) | 1.50 | 1.50 | 1.50 | 1.50 |
| | Example X | 1.50 | 1.80 | 1.80 | 1.80 |
| | TEGO ® Xymenynic (Caprylic/Capric | 1.00 | 1.00 | 1.00 | 1.00 |
| | Triglyceride, Xymenynic Acid) | | | | |
| | Diethylamino Hydroxybenzoyl Hexyl Benzoate | 6.00 | 6.00 | 6.00 | 6.00 |
| | 4-Methylbenzylidene Camphor | 4.00 | 4.00 | 4.00 | 4.00 |
| | Diethylhexyl Butamido Triazone | 3.00 | 3.00 | 3.00 | 3.00 |
| | Xanthan Gum (Keltrol CG-SFT, CP Kelco) | 0.50 | 0.50 | 0.50 | 0.50 |
| B | Water | to 100 | to 100 | to 100 | to 100 |
| | Glycerin | 3.00 | 3.00 | 3.00 | 3.00 |
| C | Carbomer | 0.10 | 0.10 | 0.10 | 0.10 |
| | TEGOSOFT ® XC MB (Phenoxyethyl Caprylate) | 0.40 | 0.40 | 0.40 | 0.40 |
| D | Sodium Hydroxide (10% in water) | q.s. | q.s. | q.s. | q.s. |
| E | Preservative | q.s. | q.s. | q.s. | q.s. |
| Z | Perfume | q.s. | q.s. | q.s. | q.s. |

### Low Viscosity W/O Sun Care Lotion SPF30 PA+++

| Phase | Raw material | % w/w | % w/w |
|---|---|---|---|
| A | ABIL ® EM 90 (Cetyl PEG/PPG-10/1 Dimethicone) | 2.50 | 2.50 |
| | TEGOSOFT ® TN (C12-15 Alkyl Benzoate) | 2.00 | 3.00 |
| | Dicaprylyl Carbonate | 2.00 | 2.00 |
| | TEGOSOFT ® CT (Caprylic/Capric Triglyceride) | 1.00 | — |
| | Example X | 0.40 | 1.60 |
| | Dimethicone (DC 200, 5 cSt, Dow Corning) | 16.00 | 16.00 |
| | Titanium Dioxide (Parsol TX, DSM) | 10.00 | 10.00 |
| | Zinc Oxide (Sunclear-Z60CO, Sunjin) | 25.00 | 25.00 |
| | Hydrogenated Lecithin | 0.50 | 1.50 |
| | Dimethicone/Vinyl Dimethicone Crosspolymer and Silica | 1.00 | 1.00 |
| | (DC 9701, DowCorning) | | |
| | Nylon-12 | 1.50 | 1.50 |
| B | Water | to 100 | to 100 |
| | Butylene Glycol | 5.00 | 5.00 |
| | Sodium Chloride | 1.50 | 1.50 |
| C | Phenoxyethanol, Ethylhexylglycerin (Euxyl PE 9010, Schülke & | 0.70 | 0.70 |
| | Mayr GmbH) | | |

### Sun Care Aqua Gel SPF50+, PA++++

| Phase | Raw material | % w/w |
|---|---|---|
| A | ABIL ® Care XL 80 (Bis-PEG/PPG-20/5 PEG/PPG-20/5 Dimethicone; | 3.00 |
| | Methoxy PEG/PPG-25/4 Dimethicone; Caprylic/Capric Triglyceride) | |
| | TEGOSOFT ® DEC (Diethylhexyl Carbonate) | 7.00 |
| | Dimethicone (5 cSt) | 2.00 |
| | TEGOSOFT ® CT (Caprylic/Capric Triglyceride) | 3.00 |
| | ABIL ® Wax 9801 (Cetyl Dimethicone) | 2.00 |
| | Bis-ethylhexyloxyphenol Methoxyphenyl Triazine (Tinosorb S, BASF) | 3.00 |
| | Diethylamino Hydroxybenzoyl Hexyl Benzoate (Uvinul A plus Granular, | 4.00 |
| | BASF) | |
| | Octocrylene | 5.00 |
| | Avobenzone (Parsol 1789, DSM) | 3.00 |
| | Homomenthyl Salicylate | 8.00 |
| | Ethylhexyl Salicylate | 5.00 |
| | Ethylhexyl Methoxycinnamate | 10.00 |
| | Example X | 1.50 |
| B | Water | to 100 |
| | Butylene Glycol | 3.00 |
| | Xanthan Gum (Keltrol CG-SFT, CP Kelco) | 0.20 |
| C | Sodium Acrylate/Sodium Acryloyldimethyl Taurate Copolymer (and) | 1.50 |
| | Isohexadecane (and) Polysorbate-80 (Simulium EG, SEPPIC) | |
| D | Phenoxyethanol, Ethylhexylglycerin (Euxyl PE 9010, Schülke & Mayr | 0.70 |
| | GmbH) | |

O/W Sun Care Cream with high protection SPF 50+PA+++

| | | % | % | % | % |
|---|---|---|---|---|---|
| Phase | Raw material | w/w | w/w | w/w | w/w |
| A | TEGO ® Care 450 MB (Polyglyceryl-3 | 3.00 | 3.00 | 2.50 | 2.50 |
| | Methylglucose Distearate) | | | | |
| | Potassium Cetyl Phosphate | — | 0.50 | — | — |
| | Glyceryl Stearate Citrate | — | — | 0.60 | — |
| | Sodium Stearoyl Glutamate | — | — | — | 0.70 |
| | Caprylic/Capric Triglyceride | 2.00 | 2.00 | — | — |
| | Butylene Glycol Dicaprylate/Dicaprate | — | — | 1.50 | 2.00 |
| | TEGO ® Alkanol 1618 (Cetearyl Alcohol) | 1.00 | 1.00 | 1.00 | 1.00 |
| | ABIL ® Wax 9840 (Cetyl Dimethicone) | 0.50 | 0.50 | 0.50 | 0.50 |
| | Bis-ethylhexyloxyphenol Methoxyphenyl Triazine | 2.00 | 2.00 | 2.00 | 2.00 |
| | (Tinosorb S, BASF SE) | | | | |
| | Butyl Methoxydibenzoylmethane | 3.00 | 3.00 | 3.00 | 3.00 |
| | Octocrylene | 8.00 | 8.00 | 8.00 | 8.00 |
| | Ethylhexyl Salicylate | 5.00 | 5.00 | 5.00 | 5.00 |
| | Example X | 2.60 | 1.90 | 2.80 | 2.30 |
| | C12-15 Alkyl Benzoate; Titanium Dioxide; PEG-30 | 8.90 | 8.90 | 8.90 | 8.90 |
| | Dipolyhydroxystearate; Silica; Dimethicone (T- | | | | |
| | lique AB50Si, Uni-Powder) | | | | |
| B | Water | to 100 | to 100 | to 100 | to 100 |
| | Glycerin | 3.00 | 3.00 | 3.00 | 3.00 |
| C | Phenylbenzimidazole Sulfonic Acid | 3.00 | 3.00 | 3.00 | 3.00 |
| | Tromethamine 78% | 2.00 | 2.00 | 2.00 | 2.00 |
| | Water | 4.00 | 4.00 | 4.00 | 4.00 |
| D | Acrylates/C10-30 Alkyl Acrylate Crosspolymer | 10.00 | 10.00 | 10.00 | 10.00 |
| | (2% solution in water) | | | | |
| E | Sodium Hydroxide (10% in water) | 0.40 | 0.40 | 0.40 | 0.40 |
| F | Phenoxyethanol, Ethylhexylglycerin (Euxyl PE | 0.70 | 0.70 | 0.70 | 0.70 |
| | 9010, Schülke & Mayr GmbH) | | | | |

### Sun Care Cream SPF 15

| | | % | % | % | % | % |
|---|---|---|---|---|---|---|
| Phase | Raw material | w/w | w/w | w/w | w/w | w/w |
| A | TEGO ® Care 450 MB (Polyglyceryl-3 | 3.00 | 2.50 | 2.50 | 2.50 | 2.50 |
| | Methylglucose Distearate) | | | | | |
| | Potassium Cetyl Phosphate | — | 0.80 | 0.80 | 0.80 | 0.80 |
| | TEGIN ® M Pellets MB (Glyceryl Stearate) | 1.50 | 1.50 | 1.50 | 1.50 | 1.50 |
| | TEGO ® Alkanol 1618 (Cetearyl Alcohol) | 1.50 | 1.50 | 1.50 | 1.50 | 1.50 |
| | TEGOSOFT ® CT (Caprylic/Capric | 2.00 | — | — | — | — |
| | Triglyceride) | | | | | |
| | Dibutyl Adipate | — | 5.00 | 5.00 | 5.00 | 5.00 |
| | dermofeel ® sensolv MB (Isoamyl Laurate) | 7.00 | 5.00 | 5.00 | 5.00 | 5.00 |
| | TEGOSOFT ® CR MB (Cetyl Ricinoleate) | 1.00 | 1.00 | 1.00 | 1.00 | 1.00 |
| | Example X | 3.00 | 1.00 | 1.30 | 1.40 | 1.40 |
| | Capryloyl Glycerin/Sebacic Acid Copolymer | — | 1.20 | — | — | — |
| | (LexFilm Sun Natural, Inolex) | | | | | |
| | Polyglyceryl-3 Stearate/Isostearate/Dimer | — | — | 1.00 | — | — |
| | Dilinoleate Crosspolymer | | | | | |
| | (CosmoSurf PG1-IS, SurfaTech Corp.) | | | | | |
| | Diisostearoyl Polyglyceryl-3 Dimer | — | — | — | 0.80 | — |
| | Dilinoleate (and) Caprylic/Capric | | | | | |
| | Triglyceride (SolAmaze Natural polymer, | | | | | |
| | Nouryon) | | | | | |
| | Sorbitol/Sebacic Acid Copolymer Behenate | — | — | — | — | 1.00 |
| | (Syncrowax ORM, Croda) | | | | | |
| | Bis-Ethylhexyloxyphenol Methoxyphenyl | 1.50 | 1.50 | 1.50 | 1.50 | 1.50 |
| | Triazine (Tinosorb S, BASF) | | | | | |
| | Butyl Methoxydibenzoylmethane | 1.00 | 1.00 | 1.00 | 1.00 | 1.00 |
| | Ethylhexyl Triazone | 0.75 | 0.75 | 0.75 | 0.75 | 0.75 |
| | Homomenthyl Salicylate | 1.50 | 1.50 | 1.50 | 1.50 | 1.50 |
| | Ethylhexyl Salicylate | 1.50 | 1.50 | 1.50 | 1.50 | 1.50 |
| | Octocrylene | 4.00 | 4.00 | 4.00 | 4.00 | 4.00 |
| | dermofeel ® TocoBalance | 0.70 | 0.70 | 0.70 | 0.70 | 0.70 |
| | (Tocopherol, Helianthus Annuus | | | | | |
| | (Sunflower) Seed Oil) | | | | | |
| | TEGO ® Feel C 10 (Cellulose) | 2.00 | 2.00 | 2.00 | 2.00 | 2.00 |
| | TEGO ® SP 13-1 (Poly C10-30 Alkyl | 1.50 | — | — | — | — |
| | Acrylate) | | | | | |
| | Phytosphingosine SLC (Salicyloyl | 0.10 | 0.10 | 0.10 | 0.10 | 0.10 |
| | Phytosphingosine) | | | | | |
| B | Water | to 100 | to 100 | to 100 | to 100 | to 100 |
| | Glycerin | 3.00 | 3.00 | 3.00 | 3.00 | 3.00 |
| | EDTA | 0.05 | 0.05 | 0.05 | 0.05 | 0.05 |
| | TEGO ® Cosmo C 100 (Creatine) | 0.50 | 0.50 | 0.50 | 0.50 | 0.50 |
| C | Xanthan Gum (Ketrol CG-SFT, CP Kelco) | 0.50 | 0.50 | 0.50 | 0.50 | 0.50 |
| D | TEGO ® Pep Up (Tetrapeptide-4; Glycerin) | 2.00 | 2.00 | 2.00 | 2.00 | 2.00 |
| E | Verstatil ® PC (Phenoxyethanol, Caprylyl | 1.20 | 1.20 | 1.20 | 1.20 | 1.20 |
| | Glycol) | | | | | |
| | Perfume | q.s. | q.s. | q.s. | q.s. | q.s. |
| | Tromethamine (Trisaminomethane, 30% in | q.s. | q.s. | q.s. | q.s. | q.s. |
| | water) | | | | | |

### W/O Foundation

| Phase | Raw material | % w/w | % w/w |
|---|---|---|---|
| A | ISOLAN ® GPS | 4.00 | — |
| | (Polyglyceryl-4 Diisostearate/Polyhydroxystearate/Sebacate) | | |
| | Polyglyceryl-6 Polyhydroxystearate; Polyglyceryl-6 Polyricinoleate | — | 3.80 |
| | (Emulium Illustro, Gattefossé) | | |
| | TEGOSOFT ® AC MB (Isoamyl Cocoate) | 5.50 | 5.50 |
| | TEGOSOFT ® DC MB (Decyl Cocoate) | 7.50 | 7.50 |
| | TEGOSOFT ® CT (Caprylic/Capric Triglyceride) | 3.50 | 3.50 |
| | CI 77891, Hydrogenated Lecithin (Unipure White LC 981 HLC, | 15.00 | 15.00 |
| | Sensient) | | |
| | CI 77492, Hydrogenated Lecithin (Unipure Yellow LC 182 HLC, | 1.20 | 1.20 |
| | Sensient) | | |
| | CI 77491, Hydrogenated Lecithin (Unipure Red LC 381 HLC Sensient) | 0.70 | 0.70 |
| | CI 77499, Hydrogenated Lecithin (Unipure Black LC 989 HLC, | 0.20 | 0.20 |
| | Sensient) | | |
| | Example X | 1.50 | 1.50 |
| B | Water | to 100 | to 100 |
| | Glycerin | 3.00 | 3.00 |
| | Zinc Sulfate Heptahydrate | 2.00 | 2.00 |
| | Verstatil ® SL non GMO (Sodium Levulinate, Potassium Sorbate, | q.s. | q.s. |
| | Water) | | |
| | Citric Acid (10% in water) | q.s. to | q.s. to |
| | | pH 4.4 | pH 4.4 |

### W/O Foundation

| Phase | Raw material | % w/w |
|---|---|---|
| A | ISOLAN ® 17 MB (Polyglyceryl-4 Diisostearate/Polyhydroxystearate/ | 4.00 |
| | Sebacate; Caprylic/Capric Triglyceride; Polyglyceryl-3 Oleate; Diisostearoyl | |
| | Polyglyceryl-3 Dimer Dilinoleate) | |
| | Isododecane | 3.33 |
| | TEGOSOFT ® XC MB (Phenoxyethyl Caprylate) | 3.33 |
| | Dimethicone | 3.33 |
| | Example X | 2.00 |
| | CI 77891, Titanium Dioxide, Alumina, Triethoxycaprylylsilane | 4.00 |
| | (Hombitan AC360, Sachtleben) | |
| | CI 77491, CI 77492, CI 77499 | 2.50 |
| | (Iron Oxide, Sicovit Brown 70 E 172, Rockwood Pigments) | |
| | Talc | 2.00 |
| B | Dicaprylyl Carbonate, Stearalkonium Hectorite, Propylene Carbonate | 3.00 |
| | (Cosmedia Gel CC, BASF SE) | |
| | Isododecane | 1.50 |
| | TEGOSOFT ® XC MB (Phenoxyethyl Caprylate) | 1.50 |
| | Dimethicone | 1.50 |
| C | Glycerin | 5.00 |
| | Magnesium Sulfate Heptahydrate | 1.50 |
| | Water | to 100 |
| D | Verstatil ® PC (Phenoxyethanol, Caprylyl Glycol) | 1.00 |
| | Boron Nitride (Boroneige 1201, ESK) | 5.00 |

SPF 30 Sun Lotion with Water-Soluble Emulsifier and Natural, Aqueous Thickeners

| | | % | % | % | % | % | % | % | % |
|---|---|---|---|---|---|---|---|---|---|
| Phase | Raw material | w/w | w/w | w/w | w/w | w/w | w/w | w/w | w/w |
| A | TEGIN ® M Pellets MB | 0.5 | 0.5 | 0.5 | 0.5 | 0.5 | 0.5 | 0.5 | 0.5 |
| | (Glyceryl Stearate) | | | | | | | | |
| | TEGO ® Alkanol 1618 | 0.5 | 0.5 | 0.5 | 0.5 | 0.5 | 0.5 | 0.5 | 0.5 |
| | (Cetearyl Alcohol) | | | | | | | | |
| | TEGOSOFT ® XC MB | 4.3 | 4.3 | 4.3 | 4.3 | 4.3 | 4.3 | 4.3 | 4.3 |
| | (Phenoxyethyl Caprylate) | | | | | | | | |
| | Example X | 1.5 | 1.5 | 1.5 | 1.5 | 1.5 | 1.5 | 1.5 | 1.5 |
| | Bis-ethylhexyloxyphenol | 3 | 3 | 3 | 3 | 3 | 3 | 3 | 3 |
| | Methoxyphenyl Triazine | | | | | | | | |
| | Butyl Methoxy | 2 | 2 | 2 | 2 | 2 | 2 | 2 | 2 |
| | Dibenzoylmethane | | | | | | | | |
| | Ethylhexyl Salicylate | 3 | 3 | 3 | 3 | 3 | 3 | 3 | 3 |
| | Octrocrylene | 8 | 8 | 8 | 8 | 8 | 8 | 8 | 8 |
| | Homosalate | 3 | 3 | 3 | 3 | 3 | 3 | 3 | 3 |
| | Ethylhexyl Triazone | 1.5 | 1.5 | 1.5 | 1.5 | 1.5 | 1.5 | 1.5 | 1.5 |
| | dermofeel ® Toco 70 non- | 0.2 | 0.2 | 0.2 | 0.2 | 0.2 | 0.2 | 0.2 | 0.2 |
| | GMO (Tocopherol, Helianthus | | | | | | | | |
| | Annuus (Sunflower) Seed Oil) | | | | | | | | |
| B | Potassium Cetyl Phosphate | 1.5 | 1.5 | 1.5 | 1.5 | 1.5 | 1.5 | 1.5 | 1.5 |
| | Glycerin | 3 | 3 | 3 | 3 | 3 | 3 | 3 | 3 |
| | Water | to 100 | to 100 | to 100 | to 100 | to 100 | to 100 | to 100 | to 100 |
| | EDTA | 0.05 | 0.05 | 0.05 | 0.05 | 0.05 | 0.05 | 0.05 | 0.05 |
| C | Xanthan Gum (Keltrol CG- | 0.3 | | | | | | | |
| | SFT, CP Kelco) | | | | | | | | |
| | Succinoglycan (Rheozan ® | | 0.3 | | | | | | |
| | SH, Solvay) | | | | | | | | |
| | Glucomannan (Rheolex ® | | | 0.3 | | | | | |
| | One-C, Nagase) | | | | | | | | |
| | Sclerotium Gum (Amigel ®, | | | | 0.4 | | | | |
| | Alban Muller) | | | | | | | | |
| | Gellan Gum (Kelcogel ® | | | | | 0.1 | | | |
| | Gellan Gum, CP Kelco) | | | | | | | | |
| | Caesalpinia Spinosa Gum | | | | | | 0.45 | | |
| | (Solagum ® Tara, Seppic) | | | | | | | | |
| | Cellulose (Exilva ® 10 wt % | | | | | | | 1 | |
| | active matter, Borregaard) | | | | | | | | |
| D | Phenoxyethanol, Caprylyl | 1 | 1 | 1 | 1 | 1 | 1 | 1 | 1 |
| | Glycol | | | | | | | | |

SPF 30 Sun Lotion with Oil-Soluble Emulsifiers and Natural, Aqueous Thickeners

| | | % | % | % | % | % | % | % | % | % | % | % | % |
|---|---|---|---|---|---|---|---|---|---|---|---|---|---|
| Phase | Raw material | w/w | w/w | w/w | w/w | w/w | w/w | w/w | w/w | w/w | w/w | w/w | w/w |
| A | TEGO ® Care PBS 6 MB | 3 | 3 | 3 | | | | | | | 3 | | |
| | (Polyglyceryl-6 Stearate, | | | | | | | | | | | | |
| | Polyglyceryl-6 Behenate) | | | | | | | | | | | | |
| | AXOL ® C 62 Pellets MB | | | | 3 | 3 | 3 | | | | | 3 | |
| | (Glyceryl Stearate | | | | | | | | | | | | |
| | Citrate) | | | | | | | | | | | | |
| | Tego ® Care 450 MB | | | | | | | 3 | 3 | 3 | | | 3 |
| | (Polyglyceryl-3 | | | | | | | | | | | | |
| | Methylglucose | | | | | | | | | | | | |
| | Distearate) | | | | | | | | | | | | |
| | TEGIN ® M Pellets MB | 0.5 | 0.5 | 0.5 | 0.5 | 0.5 | 0.5 | 0.5 | 0.5 | 0.5 | 0.5 | 0.5 | 0.5 |
| | (Glyceryl Stearate) | | | | | | | | | | | | |
| | Tego ® Alkanol 1618 | 0.5 | 0.5 | 0.5 | 0.5 | 0.5 | 0.5 | 0.5 | 0.5 | 0.5 | | 0.5 | 0.5 |
| | (Cetearyl Alcohol) | | | | | | | | | | | | |
| | TEGOSOFT ® XC MB | 4.3 | 4.3 | 4.3 | 4.3 | 4.3 | 4.3 | 4.3 | 4.3 | 4.3 | 4.3 | 4.3 | 4.3 |
| | (Phenoxyethyl | | | | | | | | | | | | |
| | Caprylate) | | | | | | | | | | | | |
| | Example X | 1.5 | 1.5 | 1.5 | 1.5 | 1.5 | 1.5 | 1.5 | 1.5 | 1.5 | 3.0 | 3.0 | 3.0 |
| | Bis-ethylhexyloxyphenol | 3 | 3 | 3 | 3 | 3 | 3 | 3 | 3 | 3 | 3 | 3 | 3 |
| | Methoxyphenyl Triazine | | | | | | | | | | | | |
| | Butyl Methoxy | 2 | 2 | 2 | 2 | 2 | 2 | 2 | 2 | 2 | 2 | 2 | 2 |
| | Dibenzoylmethane | | | | | | | | | | | | |
| | Ethylhexyl Salicylate | 3 | 3 | 3 | 3 | 3 | 3 | 3 | 3 | 3 | 3 | 3 | 3 |
| | Octrocrylene | 8 | 8 | 8 | 8 | 8 | 8 | 8 | 8 | 8 | 8 | 8 | 8 |
| | Homosalate | 3 | 3 | 3 | 3 | 3 | 3 | 3 | 3 | 3 | 3 | 3 | 3 |
| | Ethylhexyl Triazone | 1.5 | 1.5 | 1.5 | 1.5 | 1.5 | 1.5 | 1.5 | 1.5 | 1.5 | 1.5 | 1.5 | 1.5 |
| | dermofeel ® Toco 70 non- | 0.2 | 0.2 | 0.2 | 0.2 | 0.2 | 0.2 | 0.2 | 0.2 | 0.2 | 0.2 | 0.2 | 0.2 |
| | GMO (Tocopherol, | | | | | | | | | | | | |
| | Helianthus Annuus | | | | | | | | | | | | |
| | (Sunflower) Seed Oil) | | | | | | | | | | | | |
| B | Glycerin | 3 | 3 | 3 | 3 | 3 | 3 | 3 | 3 | 3 | 3 | 3 | 3 |
| | Water | to 100 | to 100 | to 100 | to 100 | to 100 | to 100 | to 100 | to 100 | to 100 | to 100 | to 100 | to 100 |
| | EDTA | 0.05 | 0.05 | 0.05 | 0.05 | 0.05 | 0.05 | 0.05 | 0.05 | 0.05 | 0.05 | 0.05 | 0.05 |
| C | Xanthan Gum (Keltrol | 0.3 | | | 0.3 | | | 0.3 | | | 0.3 | 0.3 | 0.3 |
| | CG-SFT, CP Kelco) | | | | | | | | | | | | |
| | Succinoglycan | | 0.3 | | | 0.3 | | | 0.3 | | | | |
| | (Rheozan ® SH, Solvay) | | | | | | | | | | | | |
| | Glucomannan (Rheolex ® | | | 0.3 | | | 0.3 | | | 0.3 | | | |
| | One-C, Nagase) | | | | | | | | | | | | |
| D | Phenoxyethanol, | 1 | 1 | 1 | 1 | 1 | 1 | 1 | 1 | 1 | 1 | 1 | 1 |
| | Caprylyl Glycol | | | | | | | | | | | | |

### SPF 30 Sun Lotion, Mineral UF Filter and Natural, Aqueous Thickener

| | | % | % | % | % |
|---|---|---|---|---|---|
| Phase | Raw material | w/w | w/w | w/w | w/w |
| A 1 | Tego ® Care PBS 6 MB (Polyglyceryl-6 Stearate, | 4 | | | |
| | Polyglyceryl-6 Behenate) | | | | |
| | AXOL ® C 62 Pellets MB (Glyceryl Stearate Citrate) | | 2.5 | | |
| | TEGO ® Care 450 MB (Polyglyceryl-3 Methylglucose | | | 3 | |
| | Distearate) | | | | |
| | TEGO ® Alkanol 1618 | 0.2 | 0.2 | 0.2 | 0.2 |
| | (Cetearyl Alcohol) | | | | |
| | TEGIN ® M Pellets MB | 0.2 | 0.2 | 0.2 | 0.2 |
| | (Glyceryl Stearate) | | | | |
| | Example X | 1.5 | 1.5 | 1.5 | 1.5 |
| A 2 | TEGOSOFT ® CT (Caprylic/Capric Triglyceride) | 5 | 5 | 5 | 5 |
| | dermofeel ® sensolv MB (Isoamyl Laurate) | 6 | 6 | 6 | 6 |
| | TEGOSOFT ® XC (Phenoxyethyl Caprylate) | | | | |
| | TEGOSOFT ® DEC (Diethylhexyl Carbonate) | 5 | 5 | 5 | 5 |
| | Dimethicone | 3 | 3 | 3 | 3 |
| | ABIL ® Wax 9840 (Cetyl Dimethicone) | 2 | 2 | 2 | 2 |
| | Titanium Dioxide, Silica; Dimethicone (Parsol ® TX, DSM) | 7.2 | 7.2 | 7.2 | 7.2 |
| | Zinc Oxide, Triethoxycaprylylsilane (Z-Cote ® HP 1, BASF) | 7.8 | 7.8 | 7.8 | 7.8 |
| | dermosoft ® Octiol (Caprylyl Glycol) | 0.5 | 0.5 | 0.5 | 0.5 |
| B | Potassium Cetyl Phosphate | | | | 1.5 |
| | Water | to 100 | to 100 | to 100 | to 100 |
| | Glycerin | 3 | 3 | 3 | 3 |
| | Xanthan Gum (Keltrol CG-SFT, CP Kelco) | 0.5 | 0.5 | 0.5 | 0.5 |
| D | dermosoft ® Pentiol eco (Pentylene Glycol.) | 3 | 3 | 3 | 3 |
| E | Citric Acid 50% wt.-% in water | 0.45 | 0.45 | 0.45 | 0.45 |

1. A process for producing an optionally at least partially saponified wax ester, the process comprising:
A) providing a natural wax,
B) oxidizing the natural wax to a natural wax oxidate,
C) esterifying the natural wax oxidate with at least one polyol, optionally additionally with at least one selected from monocarboxylic acids and polycarboxylic acids, and optionally
D) at least partially saponifying the wax ester obtained in C),
with the proviso that, if only one polyol is used in C), said one polyol has a hydroxyl value of less than 1240 mg KOH/g.

2. The process according to claim 1, wherein the provided natural wax is obtainable from renewable sources, said natural wax being selected from the group consisting of alfalfa wax, bamboo wax, cotton wax, beeswax, candelilla wax, caranday wax, carnauba wax, dammar wax, Douglas fir wax, esparto wax, flax wax, hemp wax, coffee wax, cork wax, oleander wax, ouricury wax, raffia wax, rice wax, rice bran wax, retamo wax, bark wax, sisal wax, tea wax, wool wax and sugarcane wax.

3. The process according to claim 1, wherein the provided natural wax contains in total 0.05% by weight to 35% by weight of aliphatic ω-hydroxycarboxylic acids and/or aliphatic α,ω-alkanediols and/or esters of aliphatic ω-hydroxycarboxylic acids and/or esters of aliphatic α,ω-alkanediols, wherein the percentages by weight refer to a total natural wax.

4. The process according to claim 1, comprising:
carrying out the oxidation in B) with chromosulfuric acid.

5. The process according to claim 1, wherein the natural wax oxidate obtained in B) has an acid value (AV) of from 30 to 200 mg KOH/g, and/or an iodine value of ≤40 g I₂/100 g, and/or a drip point of between 40° C. and 130° C.

6. The process according to claim 1, wherein the natural wax oxidate obtained in B) has a content of aliphatic monocarboxylic acids having an odd-numbered chain length of 9 to 29 carbon atoms of at least 0.01% by weight, wherein the percentages by weight refer to all aliphatic monocarboxylic acids present in the natural wax oxidate.

7. The process according to claim 1, wherein the at least one polyol has three or more carbon atoms.

8. The process according to claim 1, wherein the at least one polyol comprises polyglycerol.

9. The process according to claim 8, wherein the polyglycerol accounts for at least 20% by weight, based on all polyols.

10. The process according to claim 8, wherein the polyglycerol has a content of cyclic oligomers of from 1.0% by weight to 50% by weight.

11. An optionally at least partially saponified wax ester obtainable by a process according to claim 1.

12. A formulation, comprising:
the optionally at least partially saponified wax ester according to claim 11, and
optionally at least one substance selected from the group consisting of UV light protection filter substances, pigments, and mixtures thereof.

13. The formulation according to claim 12, wherein the optionally at least partially saponified wax ester is present in an amount of from 0.10% by weight to 20% by weight, wherein the percentages by weight refer to a total formulation.

14. (canceled)

15. A process for forming a film to boost the sun protection factor of a UV light protection filter substance or to reduce the rinseability and/or wear of a formulation from a surface the process comprising:
forming a film from the formulation according to claim 12.
